(19) United States
(12) Patent Application Publication
(10) Pub. No.: US 2026/0251609 A1
(43) Pub. Date: Aug. 27, 2026

(54) SYSTEM FOR LABEL-FREE ELECTROCHEMICAL SENSING

(71) Applicant: PRESIDENT AND FELLOWS OF HARVARD COLLEGE, Cambridge, MA (US)

(72) Inventors: Jeong-Chan LEE, Cambridge, MA (US); Pawan JOLLY, Cambridge, MA (US); Donald E. INGBER, Cambridge, MA (US); Nandhinee Radha SHANMUGAM, Cambridge, MA (US); Rohini SINGH, Cambridge, MA (US)

(73) Assignee: PRESIDENT AND FELLOWS OF HARVARD COLLEGE, Cambridge, MA (US)

(21) Appl. No.: 19/161,686

(22) PCT Filed: Mar. 11, 2024
(86) PCT No.: PCT/US2024/019384

§ 371 Date: Sep. 2, 2025

Related U.S. Application Data
(60) Provisional application No. 63/451,317, filed on Mar. 10, 2023.

(51) Int. Cl. G01N 27/327; G01N 27/414; G01N 33/543; G01N 33/58

(52) U.S. Cl. CPC G01N 27/3276; G01N 27/3271; G01N 27/3278; G01N 27/4145; G01N 33/5438; G01N 33/581

(57) ABSTRACT

The disclosure relates generally to electrodes with a target-binding molecule conjugated on its surface that is overlaid with a polymer of sufficient thickness to restrict ion mobility so that the electrical double layer is moved closer to the site of target binding to the immobilized target-binding molecule. Generally, thickness of the polymer coating layer is such that a target-binding site of the immobilized target-binding molecule is not in the coating layer, e.g., the target-binding site is exposed. The disclosure also provides sensors comprising the electrode and uses of the electrodes and sensors in target analyte detection, e.g., in label-free detection of targets. WO

### CROSS-REFERENCE TO RELATED APPLICATIONS

This application claims benefit under 35 U.S.C. § 119(e) of U.S. Provisional Application No. 63/451,317, filed Mar. 10, 2023, contents of which are incorporated herein by reference in their entirety.

### TECHNICAL FIELD

The disclosure relates generally to substrates, electrodes and sensors comprising polymeric coating layer and a target-binding molecule on a surface thereof, and uses thereof, e.g., for target analyte detection.

### BACKGROUND

Conventional label-based sensing mostly employs sandwich assays, which requires multiple reagents and detection steps for quantification of a target biomarker. In addition, these systems rely on the intrinsic properties of labels that could potentially interfere with the detection of target analyte. Label-free sensing offers plethora of opportunities to tackle these challenges, as these systems rely solely on the intrinsic physicochemical properties of biomolecular binding events. This has the benefit of reducing sample processing enabling direct detection of target analytes and thus, develop a simple, rapid, and portable sample-to-answer diagnostic device for various point-of-care (POC) diagnostic applications. Electronic sensors, in particular, electrochemical sensors are most suited for POC applications due to ease of miniaturization, scalability, and system integration. While many demonstrations of exciting progress in label-free electrochemical sensing have been reported in the literature, there has been limited success with market penetration and widespread adaption except for glucometers. This is because all biological samples contain a high concentration of mobile ions that screen charges from target molecule which attenuates the signal response reducing sensitivity.

In electrochemical sensors, the electrodes serve as an interface that transduces biological signals into a readable electrical signal response. Typically, the electrodes modified with biological capture probes when exposed to biological samples containing the target analyte undergoes charge perturbation, which manifests as electrical double layer (EDL) at the electrode/electrolyte interfaces. However, the mobile ions present in biological samples screen electric field from target analytes. Under physiological conditions, the Debye length is less than a nm, which is very small compared to size of an antibody (10-15 nm), or aptamer (30-base is ~10 nm). This intrinsic mismatch in dimensions between the size of capture probes and charge screening causes biomolecular binding interactions to occur outside EDL, which greatly attenuates the signal response reducing the sensitivity. Unfortunately, this poses a fundamental challenge, which have hindered the success of label-free sensing for more than three decades.

Thus, there is a need for methods, apparatus and sensors that can facilitate label-free analyte sensing. The present disclosure addresses these needs.

### SUMMARY

POC sensors demonstrate significant promise for at-home and patient-centric diagnostics. However, most of them are label-based that involves laborious steps required to be performed by trained personnel and have reduced stability. Presented herein are systems and methods for label-free electrochemical detection of biomarkers that are suitable for POC, at-home rapid diagnostics. The technology described herein uses a single-step fabrication technique that is desirable for low cost, portable readouts, and does not require trained users or specialized facilities. The technology described herein enables single-step sensor preparation process and enables direct quantification of target analyte without the need for redox labels. The sensor fabrication and assay steps require minimal reagents making it cost-effective and suitable for large scale manufacturing.

In general, various aspects described herein relate to electrodes and sensors having improved analyte response and detection characteristics in label-free detection methods.

In general, various aspects described herein relate to electrodes and sensors having improved analyte response and detection characteristics in label-free detection methods. In one aspect provided herein is an electrode. The electrode comprises a conductive substrate, a target-binding molecule immobilized on a surface of the conductive substrate, the target-biding molecule is overlaid with a polymeric coating layer, and at least a non-target binding portion of the target-binding molecule is covered by or embedded within the polymeric coating layer. It is noted that the target-binding molecule can be immobilized on the surface of the conductive substrate covalently or non-covalently. For example, the target-binding molecule can be covalently linked, e.g., via a linker, to the surface of the conductive substrate.

Without wishing to be bound by a theory, the polymeric coating layer moves the EDL away from the surface of the conductive substrate and towards the target-binding portion or site of the target-binding molecule. Stated in another way, the EDL is closer to the target-binding portion or site of the target-binding molecule when the polymeric coating layer is present relative to when the polymeric coating layer is absent. The term “electric double layer” as used herein means an electrically neutral boundary layer formed at the interface between a solid material (e.g., conductive substrate) and a liquid material (e.g., sample comprising an analyte of interest or an electrolyte solution). Without wishing to be bound by a theory, as the surface of the solid material attracts positive (or negative) ions in the liquid material so as to be positively (or negatively) charged, the charges in the liquid material are redistributed based on the Coulomb's law so that the level of negative (or positive) ions increases in the liquid material at the interface with the solid material, thereby forming the EDL.

As known in the art, the thickness of the EDL is the Debye-Hückel length (i.e., κ⁻¹). It is reciprocally proportional to the square root of the ion concentration C. In aqueous solutions it is typically on the scale of a few nanometers and the thickness decreases with increasing concentration of the electrolyte. Accordingly, the EDL can have a thickness of from few nanometers to one micrometer. For example, the EDL can have a thickness of from about 0.5 nm to about 10 nm. In some embodiments, the electric double layer can have a thickness of from about 0.5 nm to about 75 nm, e.g., from about 0.5 nm to about 5 nm, from about 0.5 nm to about 4 nm, from about 0.5 nm to about 3 nm, from about 0.5 nm to about 2.5 nm, from about 0.5 nm to about 2 nm, or from about 0.5 nm to about 1.5 nm. In some embodiments, the electric double layer can have a thickness of about 0.5 nm, about 1 nm, about 1.5 nm, about 2 nm, about 2.5 nm, about 3 nm, about 3.5 nm, about 4 nm, about 4.5 nm, about 5 nm, about 5.5 nm, about 6 nm, about 6.5 nm, about 7 nm, about 7.5 nm, about 8 nm, about 8.5 nm, about 9 nm, about 9.5 nm, or about 10 nm.

In some embodiments, a thickness of the polymeric coating layer is such that at least a portion of the target-binding portion or site of the target-binding molecule is in the EDL.

In some embodiments of the various aspects described herein, a target-binding portion or site of the target-binding molecule is not covered by or embedded within the polymeric coating layer. In other words, the polymeric coating layer does not cover a target-binding portion or site of the target-binding molecule. For example, a target-binding portion or site of the target-binding molecule can be exposed for contact outside the polymeric coating layer with an analyte.

In some embodiments, a target-binding portion or site of the target-binding molecule is at a surface of the polymeric coating layer. It is noted that, surface of the polymeric coating can be surface of a pore in the polymeric coating layer. In some embodiments, a target-binding portion or site of the target-binding molecule is at a surface of the polymeric coating layer and said surface of the polymeric coating layer is not a surface of a pore in the polymeric coating layer.

The polymeric coating layer can be porous or non-porous. Accordingly, in some embodiments of any one of the aspects described herein, the polymeric coating layer is substantially non-porous. As used herein, the term “non-porous” means the polymeric coating layer has at least 90% of its theoretical density. For example, a non-porous polymeric coating layer has a porosity of about 10% or less. In some embodiments, the non-porous polymeric coating layer has a porosity of about 9%, about 8%, about 7%, about 6%, about 5%, about 4%, about 3% about 2.5%, about 2%, about 1.5%, about 1%, about 0.75%, about 0.5% or lower. It is noted that porosity is inversely proportional to density of the material. The porosity of the polymeric coating layer can be determined via the ASTM F1359, ASTM F739 and/or ASTM D4284-83 standard.

In some embodiments of any one of the aspects described herein, a thickness of the polymeric coating layer is less than or equal to a distance of a target-binding portion or site of the target-binding molecule from the surface on which the target-binding molecule is present. For example, a thickness of the polymeric coating layer is about 95% (e.g., about 90%, about 85%, about 80%, about 75%, about 70%, about 65%, about 60%, about 55%, about 50%, about 45%, about 40%, about 35%, about 30%, about 25%, about 20%, about 15%, about 10%) or less of a distance between a target-binding portion or site of the target-binding molecule and the surface of the conductive substrate on which the target-binding molecule is present, e.g., when the target-binding portion or site is furthest away from the surface. In some embodiments, the polymeric coating layer has thickness that is from about 5% to about 95% (e.g., from about 5% to about 90%, from about 10 % to about 85%, or from about 15% to about 80%) of a distance between a target-binding portion or site of the target-binding molecule and the surface of the conductive substrate on which the target-binding molecule is present, e.g., when the target-binding portion or site is furthest away from the surface.

In some embodiments, a thickness of the polymeric coating layer is about 95% (e.g., about 90%, about 85%, about 80%, about 75%, about 70%, about 65%, about 60%, about 55%, about 50%, about 45%, about 40%, about 35%, about 30%, about 25%, about 20%, about 15%, about 10%) or less of a length of the target-binding molecule in its fully extended conformation. It is noted that when the target-binding molecule is linked to the surface via a linker, the length of the target-binding molecule in its fully extended conformation includes the length of the linker. In some embodiments, the polymeric coating layer has thickness that is from about 5% to about 95% (e.g., from about 5% to about 90%, from about 10 % to about 85%, or from about 15% to about 80%) of a length of the target-binding molecule in its fully extended conformation.

Without wishing to be bound by a theory, inventors have discovered inter alia that thicker polymeric coating layers provide better sensitivity in detecting target analytes. Thus, in some embodiments, the thickness of the polymeric coating layer is at least about 1 nm. For example, the polymeric coating layer has a thickness of about 1.5 nm, about 2 nm, about 2.5 nm, about 3 nm, about 2.5 nm, about 4 nm, about 4.5 nm, about 5 nm, about 5.5 nm, about 6 nm, about 6.5 nm, about 7 nm, about 7.5 nm, about 8 nm, about 8.5 nm, about 9 nm, about 9.5 nm or more.

In some embodiments, the thickness of the polymeric coating layer is about 20 nm or less. For example, the polymeric coating layer has a thickness of about 19.5 nm, about 19 nm, about 18.5 nm, about 18 nm, about 17.5 nm, about 17 nm, about 16.5 nm, about 16 nm, about 15.5 nm, about 15 nm, about 14.5 nm, about 14 nm, about 13.5 nm, about 13 nm, about 12.5 nm, about 12 nm, about 11.5, about 11 nm, about 10.5 nm, about 10 nm, about 9.5 nm, about 9 nm, about 8.5 nm, about 8 nm, about 7.5 nm, about 7 nm, about 6.5 nm, about 6 nm, about 5.5 nm, about 5 nm, about 4.5 nm, about 4 nm, about 3.5 nm, about 3 nm, about 2.5 nm, about 2 nm, about 1.5 or less. In some embodiments, the polymeric coating layer has a thickness of from about 1 nm to about 10 nm, e.g., from about 1 nm to about 9.5 nm.

Generally, the polymeric coating layer covers at least about 90% (e.g., about 91%, about 92%, about 93%, about 94%, about 95%, about 96%, about 97%, about 97%, about 99%) or more of the surface of the conductive substrate. For example, the polymeric coating layer completely covers, i.e., 100% of the surface of the conductive substrate.

Without wishing to be bound by a theory, the polymeric coating layer reduces or inhibits mobility of ions in a liquid solution to the surface of conductive substrate when the electrode is disposed in said liquid solution. For example, the polymeric coating layer can reduce or inhibit direct contact between ions in a liquid solution and the surface of the conductive substrate when the electrode is disposed in said liquid solution. In other words, the polymeric coating layer can act as an insulator or insulating layer between the target-binding portion or site of the target-binding molecule and the surface of the conductive substrate.

Also provided herein is a method for preparing an electrode described herein. The method comprises: immobilizing a target binding molecule on a surface of a conductive substrate; and forming or depositing a polymeric coating layer on said surface of the conductive substrate, and wherein the polymeric coating layer covers at least a non-target binding portion of the target-binding molecule. For example, a target-binding portion or site of the target-binding molecule is not covered by or embedded within the polymeric coating layer. The polymeric coating layer can be formed or deposited on the surface by any method. Such methods include, but are not limited electro-polymerization, photo-polymerization, auto-polymerization, and the like. In some embodiments, the step of forming or depositing the polymeric coating layer on the surface of the conductive substrate comprises electro-polymerization. In some embodiments, the step of forming or depositing the polymeric coating layer on the surface of the conductive substrate comprises photo-polymerization. In some embodiments, the step of forming or depositing the polymeric coating layer on the surface of the conductive substrate comprises auto-polymerization.

In some embodiments, the step of immobilizing the target binding molecule on the surface of the conductive substrate comprises covalently linking the target binding molecule on the surface of the conductive substrate. In some embodiments, the step of immobilizing the target binding molecule on the surface of the conductive substrate comprises linking the target binding molecule to the surface of the conductive substrate via a linker.

In another aspect, provided herein is a substrate comprising a target-binding molecule immobilized on a surface of the substrate, the target-binding molecule capable of binding with a target molecule, and a polymeric coating layer on said surface of the substrate. Generally, at least a portion (e.g., a non-target binding portion) of the target-binding molecule is covered by or embedded within the polymeric coating layer. For example, the non-target binding portion the target-binding molecule can be covered by or embedded in the polymeric layer while the target binding site of the target-binding molecule is outside the polymeric layer. Alternatively, both the non-target binding portion and the target-binding site of the target-binding molecule can be covered by or embedded within the polymeric layer. Thus, in some embodiments of any one of the aspects described herein, at least a non-target binding portion the target-binding molecule is covered by or embedded within or is inside the polymeric layer. In some embodiments, both the non-target binding portion and the target-binding site of the target-binding molecule are covered by or embedded within or are inside the polymeric layer, i.e., the target-binding molecule is encompassed in the polymeric layer. Optionally, the thickness of the polymeric coating layer is sufficient to restrict ion mobility such that the EDL is moved closer to the site of target molecule binding to the immobilized target-binding molecule on the surface of the substrate.

It is noted that the polymeric coating layer is not a molecularly imprinted polymer or layer.

In some embodiments of any one of the aspects described herein, the substrate is an electrically conductive substrate. Accordingly, in another aspect, provided herein is an electrode. The electrode comprises: (i) a conductive substrate (e.g., an electrically conductive substrate); (ii) a target-binding molecule immobilized on a surface of the conductive substrate, and the target-binding molecule capable of binding with a target analyte of interest; and (iii) and a polymeric coating layer on said surface of the conductive substrate, and wherein at least a portion (e.g., a non-target binding portion) of the target-binding molecule is embedded within the polymeric coating layer. In some embodiments of the electrode, the non-target binding portion and the target-binding site of the target-binding molecule are embedded within the polymeric layer. Optionally, the thickness of the polymeric coating layer is sufficient to restrict ion mobility such that the EDL is moved closer to the site of target molecule binding to the immobilized target-binding molecule on the surface of the conductive substrate.

In some embodiments of any one of the aspects described herein, the target-binding molecule is covalently conjugated with the surface. For example, the target-binding molecule is covalently conjugated with the surface via a linker. In some embodiments of any one of the aspects described herein, the target-binding molecule is covalently conjugated with the surface and the target-binding molecule and the polymeric coating layer are not covalently linked to each other. Target-binding molecule can be a peptide, polypeptide, a nucleic acid (e.g., oligonucleotide or polynucleotide) or a small molecule. Some exemplary target-binding molecules include, but are not limited to, receptors, ligand for a receptor, antibodies, antigen binding fragment of an antibody, antigens, enzymes, aptamers, affimers, and nucleic acids. In some embodiments of any one of the aspects described herein, the target-binding molecule is an antibody or an antigen binding fragment of an antibody.

In some embodiments of any one of the aspects described herein, the target-binding molecule and the polymer in the polymeric coating layer are not covalently linked to each other. In some embodiments, the target-binding molecule and the polymer in the polymeric coating layer are covalently linked to each other, e.g., via a linker.

Generally, the polymeric coating layer comprises an electropolymerized polymer, photopolymerized polymer, or auto-polymerized polymer. Accordingly, in some embodiments, the polymeric coating layer comprises an electropolymerized polymer. In some embodiments, the polymeric coating layer comprises an auto-polymerized polymer. In some embodiments, the polymeric coating layer comprises a photopolymerized polymer.

In some embodiments of any one of the aspects described herein, the polymeric coating layer comprises a polymer selected from the group consisting of polysaccharides, polydopamines, poly-p-phenylenes, polypyrroles, coumarin based polymers, polyhydroxyethylmethacrylates, polyindolyenes, polyanilines, polythiophenes, poly(ethylenedioxy) thiophene, poly(heteroaromatic vinylenes), polyvinylphosphates, poly-p-phenylene sulfides, poly(2,5-thienylenes), poly-o-phenylenes, poly(o-phenylene diamines), polysaccharides, polyacetylenes, porphyrins, phthalocyanins, fullerenes, polyparaphenylenes, polyphenylenevinylenes, polyfluorenes, polypyridines, polycarbazoles, polypyridinevinylenes, polyarylvinylenes, poly (p-phenylmethylvinylenes), derivatives and co-polymers thereof, and any combination thereof. For example, the polymeric coating layer comprises polydopamine, chitosan, or scopoletin (6-methoxy-7-hydroxycoumarin). In some embodiments of any one of the aspects described herein, the polymeric coating layer comprises electropolymerized polydopamine. In some embodiments, the polymeric coating layer comprises autopolymerized chitosan. In some embodiments, the polymeric coating layer comprises electropolymerized chitosan. In some embodiments, the polymeric coating layer comprises electropolymerized scopoletin.

In some embodiments of any one of the aspects described herein, the polymeric coating layer further comprises an antifouling material. Some exemplary antifouling materials include, but are not limited to, ethanolamine (ETA), hyaluronic acid (HA), and poly vinyl alcohol (PVA).

In some embodiments of any one of the aspects described herein, the polymeric coating layer further comprises a conductive element. In some other embodiments, of any one of the aspects described herein, the polymeric coating layer does not comprise a conductive element.

In some embodiments of any one of the aspects described herein, the polymeric coating layer comprises a redox active material. Some, exemplary redox active materials include, but are not limited to, metallocenes, metallocence derivatives, quinones, polycyclic aromatic hydrocarbons, redox-active organic molecules, phenazines, organosulfur compounds, radical acceptors, or a combination thereof. In some embodiments, the redox active material is poly luminol, methylene blue or ferrocene.

It is noted that the degradable materials can be included in the polymeric coating to create porous 3D matrix, such as dissolvable polymers. Integration of dissolving polymers can also be utilized to increase the porosity of the polymeric coating layer (e.g., polymers that dissolve in water (salt crystals); other can be removed by degradation (e.g., proteins or protein aggregates); temperature dependent removal (e.g., poly(N-isopropyl acrylamide) (PNIPAAm)). Some exemplary degradable polymers include, but are not limited to, poly(N-isopropyl acrylamide) (PNIPAAm), polyethylene glycol (PEG), alginate, polytetrafluoroethylene (PTFE), polyacetylene (PA), polyaniline (PANI), polypyrrole (PPy), polythiophene (PTH), poly(para-phenylene) (PPP), poly(phenylenevinylene) (PPV), and polyfuran (PF).

In some embodiments of any one of the aspects described herein, the polymeric coating layer has a porosity of about 5% to about 95%. For example, the polymeric coating layer has a porosity of about 20% to about 75%. In some embodiments, the polymeric coating layer has a porosity of about 25% to about 60%, or about 30% to about 50%. For example, the polymeric coating layer has a porosity of about 35% to 45%.

In some embodiments of any one of the aspects described herein, the polymeric coating layer comprises macropores. In some embodiments of any one of the aspects described herein, the polymeric coating layer comprises mesopores. In some embodiments of any one of the aspects described herein, the polymeric coating layer comprises nanopores. In some embodiments of any one of the aspects described herein, the polymeric coating layer comprises multiscale pores, i.e., both macropores and mesopores.

In still another aspect, provided herein is a sensor comprising a surface or electrode described herein. Generally, the sensor comprises a fluid-contact surface and the electrode is immobilized on at least a portion of the fluid-contact surface. In some embodiments of any one of the aspects described herein, the fluid-contact surface further comprises a positive control electrode and/or a negative control electrode immobilized thereon. In some embodiments of any one of the aspects described herein, the sensor comprises one or more microfluidic flow cells.

In yet another aspect, provided herein is a field-effect transistor (FET) comprising a surface or electrode described herein. Generally. The FET comprises a source electrode, a drain electrode, and a substrate described herein connecting the source electrode to the drain electrode. Also provided herein is a sensor comprising the FET.

The electrodes, surfaces and sensors described herein are useful for detecting a target analyte in a sample. Accordingly, in yet another aspect, provided herein is a use of a surface, an electrode or sensor described herein for detecting a target analyte in a sample.

In still another aspect, provided herein is a method for detecting a target analyte in a sample. Generally, the method comprises contacting a sample suspected of comprising a target analyte with an electrode or sensor described herein and detecting binding of the target analyte with the target binding molecule. Optionally, detecting the binding of the target molecule with the target binding molecule comprises applying a voltage to the electrode, and measuring a current generated from the electrode. In some embodiments, detecting the binding of the target molecule with the target binding molecule comprises label-free detection.

In some embodiments of any one of the aspects described herein, the target analyte is a peptide, a polypeptide, a peptidomimetic, a nucleic acid, an oligosaccharide, a polysaccharide, an amino acid, nucleoside, a nucleotide, a carbohydrate, a lipid, a peptidoglycan, a cell, microbial matter, an antigen, a lipid, a steroid, a hormone, a lipopolysaccharide, an endotoxin, a therapeutic agent, a lipid-binding molecule, a cofactor, a small molecule, a toxin, a biological threat agent (e.g., spore, viral, cellular and protein toxin), or any combination thereof.

The sample suspected of comprising the target analyte can be a biological sample (e.g., blood, saliva, amniotic fluid, sputum, urine, semen, cerebrospinal fluid, bronchial aspirate, perspiration, mucus, liquefied stool sample, synovial fluid, lymphatic fluid, tears, tracheal aspirate, lactation product, breath condensate and any combination thereof); or a food, an ingredient for preparing a food, poultry, meat, fish, beverage, grains, crops, or dairy product; or a non-biological sample (e.g., water, salt water, pond water, river water, reservoir water, brine, drinking water, industrial water, brown water, waste water, sewerage, soil, and mixtures thereof.

In another aspect, provided herein is a kit comprising a surface, electrode or sensor described herein.

### BRIEF DESCRIPTION OF THE DRAWINGS

This patent or application file contains at least one drawing executed in color. Copies of this patent or patent application publication with color drawings will be provided by the Office upon request and payment of the necessary fee.

FIGS. 1A-1B are schematics of interactions between the capture probe and target analyte occurring (FIG. 1A) outside EDL leading to charge-screening at unpolymerized sensor surfaces and (FIG. 1B) within EDL eliminating charge screening in electropolymerized sensor surfaces.

FIG. 2 shows bar plots demonstrating the ratio between polymerization and control (Without Polymerization). The signal response for electropolymerized sensors is ~2 times the control.

FIGS. 3A and 3B are schematic representation of potential distribution at the electrode/electrolyte interface and target analyte between (FIG. 3A) conventional and (FIG. 3B) a surface modified label-free electrochemical biosensor according to an exemplary embodiment of the disclosure. To generate higher electrical signal response, ψ_{target} and ψ_{electrode} must interact. In conventional biosensors, charge screening reduces ψ_{target} causing target analyte binding interactions to occur far from EDL whereas the novel surface-engineered sensor construct according to exemplary embodiment allows these interactions to occur within the EDL enhancing output electrical signal response.

FIGS. 4A-4C show electrochemical characterization of polydopamine in 5 mM potassium ferricyanide/ferrocyanide. (FIG. 4A) Cyclic voltammogram for polydopamine modified electrode surfaces showing decrease in peak current with increase in number of scans indicative of insulative film formation at the working electrode. Evaluation of immunosensor performance with 10 ng/ml of MIP-1β as target analyte on polydopamine modified electrode surfaces. Cyclic voltammograms showing change in signal response for (FIG. 4B) specific interaction between anti-MIP-1β capture and MIP-1β antigen, and (FIG. 4C) non-specific protein adsorption on polydopamine surface only.

FIGS. 5A-5C show electrochemical characterization of chitosan in 5 mM potassium ferricyanide/ferrocyanide. (FIG. 5A) Cyclic voltammogram for chitosan modified electrode surfaces showing decrease in peak current with increase in number of scans indicative of insulative film formation at the working electrode. Evaluation of immunosensor performance with 50 ng/ml of IL-6 as a target analyte on chitosan modified electrode surfaces. Cyclic voltammograms showing change in signal response for (FIG. 5B) specific interaction between anti-IL-6 capture and IL-6 antigen, and (FIG. 5C) non-specific protein adsorption on chitosan surface only.

FIG. 6 shows polymerization of scopoletin through cyclic voltammetry reveals the presence of two redox peaks, indicating the stable formation of scopoletin on the electrode surface.

FIG. 7 shows measurement of the open circuit potential (OCP) for the assessment of the thermodynamic stability of the polymer formed on the Au electrode. It was observed that within 5 minutes in cycles 2, 3, and 4, the OCP converged within the millivolt range, indicating a high level of stability.

FIGS. 8A and 8B show results of scopoletin-based label-free assay performance using MIP-1β spiked in human plasma specific (FIG. 8A) and non-specific (FIG. 8B) MIP-1β assay. Through EIS measurements, the specific frequency range corresponding to the antibody-antigen reaction can be extracted.

FIGS. 9A-9C show impedance changes corresponding to target binding in the MIP assay. In specific samples (FIG. 9A), significant impedance variations were observed, whereas in non-specific samples lacking capture Ab (FIG. 9B) or target MIP (FIG. 9C), no noticeable impedance changes were observed.

FIG. 10 shows impedance changes based on the presence of scopoletin. It can be noted that scopoletin reduces charge screening and exhibits a high specific signal for MIP.

FIG. 11 shows quantification of impedance changes at 10 Hz. The specific sample demonstrates a signal approximately 7 times higher than the non-specific sample.

FIG. 12 shows results of MIP assay through aging the scopoletin solution at 55° C. Signal response was similar to that of the solution stored for 25 days at room temperature.

FIG. 13 shows results of MIP assay using 0.1 mM scopoletin in 1% DMSO in DI water. No cross-reactivity was observed with TIMP capture Ab, and the highest specific signal was observed at cycle 5.

FIG. 14A is a schematic illustration for electrochemical copolymerization of dopamine and hyaluronic acid.

FIGS. 14B and 14C show results of protein adsorption test performed with 10 ng/ml of MIP-1β on (FIG. 14B) polydopamine/ETA and (FIG. 14C) polydopamine/HA modified working electrode surfaces.

FIG. 15A is a schematic illustration for electrochemical copolymerization of chitosan and poly vinyl alcohol.

FIGS. 15B and 15C show results of protein adsorption test performed with 50 ng/ml of IL-6 in plasma on (FIG. 15B) chitosan and (FIG. 15C) chitosan+PVA modified working electrode surfaces (data shown for 3 cycles).

### DETAILED DESCRIPTION

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference in their entirety for any purpose.

### Polymeric Coating Layer

Generally, the polymeric coating layer comprises a polymer. In some embodiments, the polymer coating layer is non-conductive. It is noted that a polymer comprised in the polymeric coating layer can be an electropolymerized polymer, photopolymerized polymer, or auto-polymerized polymer. In some embodiments of any one of the aspects described herein, the polymeric coating layer comprises an electropolymerized polymer. In some embodiments of any one of the aspects described herein, the polymeric coating layer comprises an auto-polymerized polymer. In some embodiments of any one of the aspects described herein, the polymeric coating layer comprises a photopolymerized polymer.

Exemplary polymers for the polymeric coating layer include, but are not limited to, polysaccharides, polydopamines, poly-p-phenylenes, polypyrroles, coumarin based polymers, polyhydroxyethylmethacrylates, polyindolyenes, polyanilines, polythiophenes, poly(ethylenedioxy) thiophene, poly(heteroaromatic vinylenes), polyvinylphosphates, poly-p-phenylene sulfides, poly(2,5-thienylenes), poly-o-phenylenes, poly(o-phenylene diamines), polysaccharides, polyacetylenes, porphyrins, phthalocyanins, fullerenes, polyparaphenylenes, polyphenylenevinylenes, polyfluorenes, polypyridines, polycarbazoles, polypyridinevinylenes, polyarylvinylenes, poly (p-phenylmethylvinylenes), derivatives and co-polymers thereof, and any combination thereof.

In some embodiments of any one of the aspects described herein, the polymeric coating layer comprises polydopamine, chitosan, or scopoletin. For example, the polymeric coating layer comprises electropolymerized polydopamine. In another non-limiting example, the polymeric coating layer comprises auto-polymerized chitosan. In yet another example, the polymeric coating layer comprises electropolymerized chitosan. In still another example, the polymeric coating layer comprises electropolymerized scopoletin.

Generally, the polymer in the polymeric coating layer is chosen to be complementing the target-binding molecule properties. The polymer is capable of being deposited in a controlled method, is compact and is biocompatible. In some embodiments, the polymer is capable of replacing ions in the solution and/or displacing the Debye length by changing the position of Helmholtz plane.

It is noted that the polymer in the polymeric coating layer is not a proteinaceous material, i.e., the polymer is not a polypeptide.

It is noted that polymeric layer can be formed on a surface by electropolymerization. Methods for electropolymerizing polymer are well known in the art. Such methods include, but are not limited to, voltammetry, amperometry, etc. In some embodiments, the polymeric layer can be formed on a surface by performing cyclic voltammetry (CV) from −1 to +1 V (e.g., from −0.75 to +0.75 V, from −0.5 to +0.5 V or from −0.2 V to +0.5 V) at the scan rate of 5-500 m V/s (e.g., 5 mV/s, 10 mV/s, 15 mV/s, 20 mV/s, 25 mV/s, 30 mV/s, 35 mV/s, 40 mV/s, 45 m V/s, 50 m V/s, 55 m V/s, 60 m V/s, 65 m V/s, 70 mV/s, 75 m V/s, 80 mV/s, 85 mV/s, 80 mV/s, 95 m V/s, or 100 mV/s) for at least 2 (e.g., 3, 4, 5, 6, 7, 8, 9, 10, 11, 12, 13, 14, 15, 16, 17, 18, 19, 20, 21, 22, 23, 24, 25, 26, 27, 28, 29, 30 or more) cycles. The thickness of polymer layer can be controlled by altering the number of CV cycles. As CV cycling proceeds, more polymer gets deposited at the surface resulting in the formation of polymeric coating layer on the surface.

In some embodiments of any one of the aspects described herein, a thickness of the polymeric coating layer is less than or equal to a distance of a target-binding site of the target-binding molecule from the surface on which the target-binding molecule is present. For example, a thickness of the polymeric coating layer is less than or equal to a distance of a target-binding site of the target-binding molecule from the surface on which the target-binding molecule is present, when the target-binding site is furthest away from the surface.

In some embodiments of any one of the aspects described herein, a thickness of the polymeric layer is less than or equal to a length of the target-binding molecule in its fully extended conformation.

In some embodiments of the various aspects described herein, a thickness of the polymeric coating layer is equal to or greater than a Debye length of a target molecule associated with the target-binding molecule.

As used herein, “Debye length” refers to the distance from the electrode surface at which ions in a solution effectively screen an electrical response induced by the target analyte binding to the capture probe, i.e., the distance at which significant charge separation can occur. Stated in another way, Debye length is a measure of the distance that the counter-ions extend away from the electrode surface. The Debye length is calculated using the Debye-Hückel equation: ~\sqrt{εKT/C₀}, where A_{D}=Debye length, ε=electric constant, k=Boltzman constant, T=temperature, and C₀-ionic concentration. Typically, for physiological solutions, the Debye length is about 1 nm or less. This means that for binding of the target molecules to the capture probe that extend more than about 1 nm from the electrode surface into the sample volume, any electrostatic effects outside the Debye length tend to be screened or shielded from detection by the excess free charge. As is known in the art, the salt concentration has an effect on the Debye length according to the Debye-Hückel model. For example, the Debye length is about 1 nanometer (nm) for 100 millimolar (mM) potassium chloride (KCl), about 3.4 nm for 10 mM KCl and about 10 nm for 1 mM KCl.

In some embodiments of any one of the aspects, Debye length can range from about 0.1 nm to about 100 nm. For example, the Debye length can be from about 0.2 nm to about 75 nm, from about 0.3 nm to about 50 nm, from about 0.4 nm to about 25 nm, from about 0.5 nm to about 20 nm, or from about 0.75 nm to about 15 nm. In some embodiments of any one of the aspects, Debye length can be from about 1 nm to about 10 nm.

The polymeric coating layer can have a thickness of from about 0.1 nm to about 200 μm. In some embodiments of any one of the aspects described herein, the polymeric coating layer can have a thickness of from about 0.1 nm to about 500 nm. For example, the polymeric coating layer can have a thickness of from about 0.5 nm to about 250 nm, from about 1 nm to about 200 nm or from about 1.5 nm to about 150 nm. In some embodiments, the polymeric coating layer can have a thickness of from about 2 nm to about 100 nm. In some embodiments, the polymeric coating layer can have a thickness of from about 1 nm to about 20 nm. For example, the polymeric coating layer can have a thickness of from about 1 nm to about 10 nm.

In some embodiments of any one of the aspects described herein, the polymeric coating layer can have a thickness of from about 0.5 μm to about 175 μm. For example, the polymeric coating layer can have a thickness of from about 0.75 μm to about 150 μm, from about 1.5 μm to about 100 μm, from about 2 μm to about 75 μm., or from about 2.5 μm to about 50 μm. In some embodiments, the polymeric coating layer can have a thickness of from about 5 μm to about 25 μm.

In some embodiments of any one of the aspects described herein, the polymeric coating layer is porous. As used herein, the term “porous” in the context polymeric coating layer means the polymeric coating layer comprises a plurality of spores, holes, openings, bores, apertures, spaces, perforations, or intervals. While the term porous indicates the presence of voids, it does not specify the specific size of the spores, holes, openings, bores, apertures, spaces, perforations, or intervals. The term “porosity” is widely understood as the ratio of void volume to total volume of a three-dimensional porous body, where the total volume is determined by the macroscopic outer dimensions of the body. Porosity can be indicated as a fraction between 0-1 or as a percentage between 0-100%. Porosity can be measure by instruments in the art, such as a porometer. Porosity is inversely proportional to density of the material. Thus, the porosity also can be determined by measuring the density of the coating layer. In some embodiments of any one of the aspects described herein, the porosity can be determined by mercury porosimetry analysis. In some embodiments, mercury porosimetry analysis corresponds to the intrusion of a volume of mercury characteristic of the existence of pores in the polymeric coating layer according to the ASTM D4284-83 standard.

Generally, the polymeric coating layer has a porosity from about 5% to about 95%. For example, the polymeric coating layer has a porosity from about 10% to about 75%, about 15% to about 70%, about 20% to about 65%, about 25% to about 60%, or about 30% to about 55%. In some embodiments of any one of the aspects described herein, the polymeric coating layer has a porosity from about 35% to about 45%.

In some embodiments of any one of the aspects described herein, the polymeric coating layer comprises macropores. As used herein, the term “macropore” means pores whose aperture, width or diameter is greater than 100 nm. In some embodiments of any one of the aspects described herein, macropores have an aperture, width or diameter from about 0.1 μm to about 10 μm. For example, macropores have an aperture, width or diameter from about 0.25 μm to about 7.5 μm, from about 0.5 μm to about 5 μm, from about 0.75 μm to about 2.5 μm, or from about 1 μm to about 3 μm. In some embodiments of any one of the aspects described herein, macropores have an aperture, width or diameter of about 0.1 μm, about 0.15 μm, about 0.2 μm, about 0.25 μm, about 0.3 μm, about 0.35 μm, about 0.4 μm, about 0.45 μm, about 0.5 μm, about 0.55 μm, about 0.6 μm, about 0.65 μm, about 0.7 μm, about 0.75 μm, about 0.8 μm, about 0.85 μm, about 0.9 μm, about 0.95 μm, about 1 μm, about 1.05 μm, about 1.1 μm, about 1.15 μm, about 1.2 μm, about 1.25 μm, about 1.3 μm, about 1.35 μm, about 1.4 μm, about 1.45 μm, about 1.5 μm, about 1.55 μm, about 1.6 μm, about 1.65 μm, about 1.7 μm, about 1.75 μm, about 1.8 μm, about 1.85 μm, about 1.9 μm, about 1.95 μm, about 2 μm, about 2.05 μm, about 2.1 μm, about 2.15 μm, about 2.2 μm, about 2.25 μm, about 2.3 μm, about 2.35 μm, about 2.4 μm, about 2.45 μm, about 2.5 μm, about 2.55 μm, about 2.6 μm, about 2.65 μm, about 2.7 μm, about 2.75 μm, about 2.8 μm, about 2.85 μm, about 2.9 μm, about 2.95 μm, about 3 μm, about 3.05 μm, about 3.1 μm, about 3.15 μm, about 3.2 μm, about 3.25 μm, about 3.3 μm, about 3.35 μm, about 3.4 μm, about 3.45 μm, about 3.5 μm, about 3.55 μm, about 3.6 μm, about 3.65 μm, about 3.7 μm, about 3.75 μm, about 3.8 μm, about 3.85 μm, about 3.9 μm, about 3.95 μm, about 4 μm, about 4.05 μm, about 4.1 μm, about 4.15 μm, about 4.2 μm, about 4.25 μm, about 4.3 μm, about 4.35 μm, about 4.4 μm, about 4.45 μm, about 4.5 μm, about 4.55 μm, about 4.6 μm, about 4.65 μm, about 4.7 μm, about 4.75 μm, about 4.8 μm, about 4.85 μm, about 4.9 μm, about 4.95 μm, or about 5 μm.

In some embodiments of any one of the aspects described herein, the polymeric coating layer comprises mesopores. As used herein, the term “mesopores” means pores whose aperture, width or diameter is between about 5 nm and about 99 nm. In some embodiments of any one of the aspects described herein, mesopores have an aperture, width or diameter from about 5 nm to about 50 nm. For example, mesopores have an aperture, width or diameter of about 5 nm, about 5.5 nm, about 6 nm, about 6.5 nm, about 7 nm, about 7.5 nm, about 8 nm, about 8.5 nm, about 9 nm, about 9.5 nm, about 10 nm, about 10.5 nm, about 11 nm, about 11.5 nm, about 12 nm, about 12.5 nm, about 13 nm, about 13.5 nm, about 14 nm, about 14.5 nm, about 15 nm, about 15.5 nm, about 16 nm, about 16.5 nm, about 17 nm, about 17.5 nm, about 18 nm, about 18.5 nm, about 19 nm, about 19.5 nm, about 20 nm, about 20.5 nm, about 21 nm, about 21.5 nm, about 22 nm, about 22.5 nm, about 23 nm, about 23.5 nm, about 24 nm, about 24.5 nm, about 25 nm, about 25.5 nm, about 26 nm, about 26.5 nm, about 27 nm, about 27.5 nm, about 28 nm, about 28.5 nm, about 29 nm, about 29.5 nm, about 30 nm, about 30.5 nm, about 31 nm, about 31.5 nm, about 32 nm, about 32.5 nm, about 33 nm, about 33.5 nm, about 34 nm, about 34.5 nm, about 35 nm, about 35.5 nm, about 36 nm, about 36.5 nm, about 37 nm, about 37.5 nm, about 38 nm, about 38.5 nm, about 39 nm, about 39.5 nm, about 40 nm, about 40.5 nm, about 41 nm, about 41.5 nm, about 42 nm, about 42.5 nm, about 43 nm, about 43.5 nm, about 44 nm, about 44.5 nm, about 45 nm, about 45.5 nm, about 46 nm, about 46.5 nm, about 47 nm, about 47.5 nm, about 48 nm, about 48.5 nm, about 49 nm, about 49.5 nm, or about 50 nm. In some embodiments of any one of the aspects described herein, the mesopores have an aperture, width or diameter from about 5 nm to about 20 nm. For example, the mesopores have an aperture, width or diameter from about 10 nm to about 15 nm.

In some embodiments of any one of the aspects described herein, the polymeric coating layer comprises macropores and mesopores. For example, the polymeric coating layer comprises macropores having an aperture, width or diameter from about 0.1 μm to about 10 μm, and mesopores having an aperture, width or diameter from about 5 nm to about 50 nm. In some embodiments of any one of the aspects described herein, the polymeric coating layer comprises macropores having an aperture, width or diameter from about 1 μm to about 5 μm, and mesopores having an aperture, width or diameter from about 10 nm to about 15 nm.

In some embodiments of any one of the aspects described herein, the polymeric coating layer comprises nanopores. As used herein, the term “nanopores” means pores whose aperture, width or diameter is less than about 5 nm, typically strictly greater than 0 and less than about 5 nm.

In some embodiments of any one of the aspects described herein, the polymeric coating layer comprises macropores and nanopores. For example, the polymeric coating layer comprises macropores, mesopores and nanopores.

### Target-Binding Molecule (Capture Probe)

The surfaces, electrodes and sensors described herein include a target-binding molecule. The terms “target-binding ligand”, “target-binding molecule” and “capture probe” are used interchangeably herein and refer to a molecule that binds to or interacts with a target molecule. In other words, a target-binding ligand or molecule is a molecule that is capable of binding with a target molecule. The targeting binding ligand can be a natural or synthetic molecule (e.g., a molecular receptor) that binds to a target molecule. Exemplary target-binding ligands include, but are not limited to, a receptor, a ligand for a receptor, an antibody, an antigen binding fragment of an antibody, an antigen, an enzyme, a nucleic acid, an aptamer or affimers. The target-binding ligand is also referred to as a “capture agent” or “capture molecule” herein.

In some embodiments of any one of the aspects described herein, the binding of the target-binding ligand to the target molecule is a specific binding such that it is selective to that target above non-targets. For example the dissociation constant between the target-binding ligand and target molecule is at least about 200 nM, alternatively at least about 150 nM, alternatively at least about 100 nM, alternatively at least about 60 nM, alternatively at least about 50 nM, alternatively at least about 40 nM, alternatively at least about 30 nM, alternatively at least about 20 nM, alternatively at least about 10 nM, alternatively at least about 8 nM, alternatively at least about 6 nM, alternatively at least about 4 nM, alternatively at least about 2 nM, alternatively at least about 1 nM, or greater. In certain embodiments, the specific binding refers to binding where the target-binding ligand binds to its target molecule without substantially binding to any other species in the sample/test solution.

By way of non-limiting examples, a target-binding ligand can be selected from antibodies, adnectins, ankyrins, other antibody mimetics and other protein scaffolds, aptamers, nucleic acid (e.g., an RNA or DNA aptamer), protein, peptide, binding partner, oligosaccharides, polysaccharides, lipopolysaccharides, cellular metabolites, cells, viruses, subcellular particles, haptens, pharmacologically active substances, alkaloids, steroids, vitamins, amino acids, avimers, peptidomimetics, hormone receptors, cytokine receptors, synthetic receptors, sugars or molecularly imprinted polymer. The target-binding ligand can be selective to a specific target or class of targets such as toxins and biomolecules. For example, the target can be ions, molecules, oligomers, polymers, proteins, peptides, nucleic acids, toxins, biological threat agents such as spore, viral, cellular and protein toxins, carbohydrates (e.g., mono saccharides, disaccharides, oligosaccharides, polyols, and polysaccharides) and combinations of these (e.g., copolymers including these).

In some embodiments of any one of the aspects described herein, the target-binding ligand is an antibody or antigen binding fragment thereof. As used herein, the terms “antibody” and “antibodies” include polyclonal antibodies, monoclonal antibodies, humanized or chimeric antibodies, single chain Fv antibody fragments, Fab fragments, and F(ab)₂ fragments. Antibodies having specific binding affinity for a target of interest (e.g., an antigen) can be produced through standard methods. As used herein, the terms “antibody” and “antibodies” refer to intact antibody, or a binding fragment thereof that competes with the intact antibody for specific binding and includes chimeric, humanized, fully human, and bispecific antibodies. In some embodiments, binding fragments are produced by recombinant DNA techniques. In additional embodiments, binding fragments are produced by enzymatic or chemical cleavage of intact antibodies. Binding fragments include, but are not limited to, Fab, Fab′, F(ab′)₂, Fv, and single-chain antibodies.

In some embodiments of any one of the aspects described herein, the target-binding molecule is covalently conjugated with the surface. For example, the target-binding molecule is covalently conjugated with the surface via a linker. In some embodiments of any one of the aspects described herein, the target-binding molecule is covalently conjugated with the surface and the target-binding molecule and the polymeric coating layer are not covalently linked to each other. Some exemplary target-binding molecules include, but are not limited to, receptors, ligand for a receptor, antibodies, antigen binding fragment of an antibody, antigens, enzymes, and nucleic acids. In some embodiments of any one of the aspects described herein, the target-binding molecule is an antibody or an antigen binding fragment of an antibody.

### Antifouling Material

Embodiments of the various aspects described herein include an antifouling material. As used herein an antifouling material is a molecule, substance or composition that inhibits, prevents or reduces non-specific adsorption of molecules, e.g., target molecules on a surface, e.g., the polymeric coating. Some exemplary antifouling materials include, but are not limited to, ethanolamine (ETA), hyaluronic acid (HA), and poly vinyl alcohol (PVA).

### Conductive Element

Embodiments of the various aspects described herein include a conductive element. As used herein a conductive element is a substance or substrate that has the capability to conduct electricity. The conductive element can comprise conducting and/or semi-conducting materials. Further, the conductive element can be in any desired shape or form. For example, the conductive element can be in form of particles (e.g., nanoparticles), rods, flakes (e.g., nanoflakes), tubes (e.g., nanotubes), fibers, sheets, films, and the like. For example, the conductive element can be included in the form of a particle, a nano-particle, a micro-particle, a fiber, a nano-fiber, a micro-fiber, a flake, a nanoflake, a microflake, a tube, a nanotube, a microtube, a crystal, a nanocrystal, a microcrystal, a wire, a nano-wire, a micro-wire, a rod, a nano-rod, a micro-rod, a foil, a sheet, a web, or any combinations of these forms.

The conductive element can be formed from one or more metals, e.g., copper, gold, silver, platinum, palladium, indium, iridium, rhodium, ruthenium, osmium, nickel, tin, titanium, tantalum, tungsten, chromium, iron, aluminum, zinc, combinations thereof, or alloys of any of the foregoing. In addition, or in the alternative, a nonmetallic conductive material can be used. Exemplary nonmetallic conductive materials include, but are not limited to, graphite or acetylene black, graphene, conductive ceramics such as indium tin oxide (ITO), titanium nitride, tungsten nitride, tantalum nitride, and conductive polymers such as polythiophenes, polyanilines, polypyrroles, and polyetheylenes and their mixtures and derivatives.

In some embodiments of any one of the aspects, the conductive element comprises a metal or a metalloid. For example, the conductive element comprises gold. In some embodiments of any one of the aspects described herein, the conductive element comprises gold particles (e.g., gold nano-particles), gold wires (e.g., gold nanowires), gold rods (e.g., gold nano-rods), or any combinations thereof.

In some embodiments of any one of the aspects described herein, the conductive element comprises a conducting carbon-based material. For example, the conductive element comprises an allotrope of carbon atoms arranged in a hexagonal lattice. The allotropes of carbon can include some functionalization, such as oxygen, carboxylates, epoxides, amines, amides and combinations of these, as described below. In some implementations, the functionalization includes poly amine functionalization such as pentaamine functionalization. In some embodiments, the conductive element comprises graphite, graphene, graphene oxide, functionalized graphene oxide, reduced graphene oxide (rGO), functionalized reduced graphene oxide, or carbon nano-tubes (CNTs).

As used herein “carbon nanotubes” and “graphene” are allotropes of carbon with sp² carbon atoms arranged in a hexagonal, honeycomb lattice. Single layer graphene is a two-dimensional material, and is a single layer of graphite. As used herein, more than one layer of graphene can be referred to as graphene, for example between 1 and 200 layers (e.g., about 1 to 100 layers, about 1 to 50 layers, about 1 to 10 layers). Carbon nanotubes are hollow, cylindrical structures, formed as a sheet of graphene rolled into a cylinder.

As used herein “graphene oxide” is a material that can be formed from the oxidation of graphene or exfoliation of graphite oxide. In a first step for producing graphene oxide, graphite is oxidized. Several methods for oxidation are known, one common method known as the Hummers and Offeman method, in which graphite is treated with a mixture of sulphuric acid, sodium nitrate and potassium permanganate (a very strong oxidizer). Other methods are known to be more efficient, reaching levels of 70% oxidization, by using increased quantities of potassium permanganate, and adding phosphoric acid combined with the sulphuric acid, instead of adding sodium nitrate. Exfoliation of graphene oxide provides graphite oxide and can be done by several methods. Sonication can be a very time-efficient way of exfoliating graphite oxide, and it is extremely successful at exfoliating graphene (almost to levels of full exfoliation), but it can also heavily damage the graphene flakes, reducing them in surface size from microns to nanometers, and also produces a wide variety of graphene platelet sizes. Mechanically stirring is a much less destructive approach, but can take much longer to accomplish.

Graphite oxide and graphene oxide are very similar, chemically, but structurally, they are very different. Both are compounds having carbon, oxygen and hydrogen in variable ratios. In the most oxidized state the oxygen amount can be as high as about 60 wt %. the amount of hydrogen varies depending on the functionalization, for example, the number of epoxy bridges, hydroxyl groups and carboxyl groups. The main difference between graphite oxide and graphene oxide is the interplanar spacing between the individual atomic layers of the compounds, caused by water intercalation. This increased spacing, caused by the oxidization process, also disrupts the sp² bonding network, meaning that both graphite oxide and graphene oxide are often described as electrical insulators.

Reduced graphene oxide (rGO) is prepared from reduction of graphene oxide by thermal, chemical or electrical treatments. For example, treating the graphene oxide with; hydrazine, hydrogen plasma, heating in water, high temperature heating (e.g., under nitrogen/argon) and electrochemical reduction. Whereas graphene can be a single carbon layer ideally comprising only carbon, reduced graphene oxide is similar but contains some degree of oxygen functionalization. The amount of oxygen depends on the degree of reduction and in some materials can vary between about 50 wt % and about 1 wt. % (e.g., between about 30 wt. % and about 5 wt. %).

Reduced graphene oxide can be functionalized or include functional groups. For example, reduced graphene oxide often includes oxygen in the form of carboxyl groups and hydroxyl groups. In some forms, the carboxyl and hydroxyl groups populate the edges of the rGO sheets, which can be functionalized. Accordingly, in some embodiments, the reduced graphene oxide (rGO) is carboxylated reduced graphene oxide or aminated reduced graphene oxide. As used herein, carbonylated reduced graphene oxide can refer to reduced graphene oxide having carboxyl groups. In some embodiments the amount of oxygen attributable to the carboxyl groups is between about 30 wt. % and about 0.1 wt. % (e.g., between about 10 wt. % and about 1 wt. %). Other forms of functionalization are possible. For example, amine functionalized rGO can be formed by a modified Buchere reaction, wherein ammonia an graphene oxide is reacted using a catalyst such as sodium bisulfite, or epoxide groups on graphene oxide can be opened with p-phenylenediamine. In some embodiments, the amount of nitrogen is between about 30 wt. % and 0.1 wt. % (e.g., between about 10 wt. % and 1 wt. %). In some implementations, a polyamine is used to functionalize rGO. For example, pentaamine functionalized graphene is used in some implementations.

The tube-shaped carbon nanotubes have diameters in the nanometer scale, such as, for example, between about 0.2 and about 20 nm, preferably between about 0.5 and about 10 nm, and more preferably still between about 1 and about 5 nm. These can be single walled carbon nanotubes (SWCNT), multi walled carbon nanotubes (MWCNT) (e.g., a collection of 2 or more nested tubes of continuously increasing diameters, or mixtures of these). The diameters of MWCNT can be larger than the SWCNT, such as between about 1 and about 100 nm (e.g., between about 1 and about 50 nm, between about 10 and 20 nm, between 5 and 15 nm, between about 30 and 50 nm). Depending on how the precursor graphene sheet is rolled up to make a seamless cylinder that is the carbon nanotube, different isomers of carbon nanotube can be made, for example designated as armchair configuration, chiral configuration, and zigzag configuration. In some embodiments, the carbon nanotubes are carboxylated carbon nanotubes or aminated carbon nanotubes.

The carbon nanotubes and reduced graphene oxide can include intercalated materials, such as ions and molecules. In some embodiments the carbon nanotubes can be functionalized for example by oxidation to form carboxylic acid groups on the surface, providing CNTs. In addition, in some embodiments, the carbon nanotubes and rGO can be further modified through condensation reactions with the carboxylic acid groups present on the CNTs or rGO (e.g., with alcohols and amines), electrostatic interactions with the carboxylic acid groups (e.g., calcium mediated coupling, or quaternary amines, protonated amine-carboxylate interaction, through cationic polymers or surfactants) or hydrogen bonding through the carboxylic acid groups (e.g., with fatty acids, and other hydrogen bonding molecules). The functionalization can be partial (e.g., wherein less than 90%, less than 80%, less than 60%, less than 50%, less than 40%, less than 30%, less than 20%, less than 10%, more than 10%, more than 20%, more than 30%, more than 40%, more than 50%, more than 60%, more than 70%, more than 80%, of the available carboxylic acid groups are functionalized) or complete, such as functionalizing substantially all the carboxylic acids (e.g., more than 90%, more than 95%, more than 99% of available carboxylic acid groups).

In some embodiments of any one of the aspects, the conductive element comprises a conductive polymer. Exemplary conductive polymers include, but are not limited to, polyacrylonitrile (PAN), polyanilines, polypyrroles, polyacetylenes, polyphenylene sulfide, polythiophene, polyfluorene, polypyrene, polyazulene, polynaphthalene, polycarbazole, polyindole, polyazepine, poly (3,4-ethylenedioxythiophene) (PEDOT), poly (p-phenylene sulfide) (PPS), poly(p-phenylene vinylene), poly(fluorenes)s, polyphenylenes, polypyrenes, polyazulenes, polynaphthalenes, polyanilines, polyazepines, polyindoles, polycarbazoles, poly(pyrrole)s, poly(thiophene)s, poly (p-phenylene vinylene) (PPV), and mixtures thereof.

In some embodiments of any one of the aspects described herein, the conductive element comprises one or more organic compounds having conducting and/or semiconducting properties. Exemplary organic compounds having conducting and/or semiconducting properties include, but are not limited to polyacetylenes, porphyrins, phthalocyanins, fullerenes, polyparaphenylenes, polyphenylenevinylenes, polyfluorenes, polythiophenes, polypyrroles, polypyridines, polycarbazoles, polypyridinevinylenes, polyarylvinylenes, poly (p-phenylmethylvinylenes), derivatives and co-polymers thereof, and any combination thereof. In some embodiments of any one of the aspects described herein, the organic compounds having conducting and/or semiconducting properties are selected from the group consisting of poly(3-hexylthiophene) (P3HT), perfluoro-3,6-dioxa-4-methyl-7-octenesulfonic acid copolymer, tetrafluoroethylene-perfluoro-3,6-dioxa-4-methyl-7-octenesulfonic acid copolymer (e.g. nafion), poly(9,9-dioctylfluorene-2,7-diyl-co-bis-N,N-(4-butylphenyl)-bis-N,N-phenyl-1,4-phenylenediamine), poly(9,9-dioctylfluorene-2,7-diyl-co-benzothiadiazole), (6,6)-phenyl-C61-butyric acid methyl ester, poly(2-methoxy-5-(2′-ethyl-hexyloxy)-1,4-phenylene vinylene), poly(4-vinylphenol) (PVP), and copolymers thereof, and any combination thereof.

The conductive element can be cross-linked with the surface and/or other components present in the in the polymeric coating layer. In some embodiments, the conductive element is cross-linked with the surface and/or another component in the polymeric coating layer by a cross-linking agent. In some embodiments, the conductive element in the polymeric coating layer is covalently linked to the surface that is coated by the polymeric coating layer.

In some embodiments of any one of the aspects described herein, the conductive element is cross-linked with another component in the polymeric coating layer by a cross-linking agent selected from Genipin, polyethylene glycol, and glutaraldehyde.

The ratio of the conductive element to the cross-linking agent can be from about 100:1 to about 1:1 (w/w). In some embodiments of any one of the aspects described herein, the ratio of the conductive element to the cross-linker is from about 100:1 to about 10:1 (w/w). For example, the ratio of the conductive element to the cross-linker can be from about 90:1 to about 20:1, about 80:1 to about 30:1, about 70:1 to about 40:1, or about 60:1 to about 50:1 (w/w). In some embodiments, the w/w ratio of the conductive element to cross-linker is about 100:1, or about 95:1, or about 90:1, or about 85:1, or about 80:1, about 75:1, or about 70:1, or about 65:1, or about 60:1, about 55:1, or about 50:1, or about 45:1, or about 40:1, about 35:1, or about 30:1, or about 25:1, or about 20:1, or about 15:1, or about 10:1, or about 5:1 or about 1:1.

The amount of the conductive element in a composition used for preparing the polymeric coating layer can range from about 1 mg/ml to about 20 mg/ml. For example, the amount of the conductive element in the composition or the polymeric coating layer can be about 1 mg/ml, about 1.5 mg/ml, about 2 mg/ml, about 2.5 mg/ml, about 3 mg/ml, about 3.5 mg/ml, about 4 mg/ml, about 4.5 mg/ml, about 5 mg/ml, about 5.5 mg/ml, about 6 mg/ml, about 6.5 mg/ml, about 7 mg/ml, about 7.5 mg/ml, about 8 mg/ml, about 8.5 mg/ml, about 9 mg/ml, about 9.5 mg/ml, about 10 mg/ml, about 10.5 mg/ml, about 11 mg/ml, about 11.5 mg/ml, about 12 mg/ml, about 12.5 mg/ml, about 13 mg/ml, about 13.5 mg/ml, about 14 mg/ml, about 14.5 mg/ml, about 15 mg/ml, about 15.5 mg/ml, about 16 mg/ml, about 16.5 mg/ml, about 17 mg/ml, about 17.5 mg/ml, about 18 mg/ml, about 18.5 mg/ml, about 19 mg/ml, about 19.5 mg/ml, or about 20 mg/ml. In some embodiments of any one of the aspects described herein, the amount of the conductive element in a composition used for preparing the polymeric coating layer is from about 2 mg/ml to about 18 mg/ml, about 3 mg/ml to about 17 mg/ml, about 4 mg/ml to about 16 mg/ml. For example, the amount of the conductive element in the composition used for preparing the polymeric coating layer is from about 5 mg/ml to about 15 mg/ml. In some embodiments, amount of the conductive element in the composition used for preparing the polymeric coating layer is from about 5 mg/ml to about 10 mg/ml.

In some embodiments, the amount of the conductive element in a composition used for preparing the polymeric coating layer or the polymeric coating layer itself can range from about 0.01 to about 20% (w/v, w/w or v/v), e.g., 0.01% to about 10% (w/v, w/w or v/v). For example, the amount of the conductive element in a composition used for preparing the polymeric coating layer or the polymeric coating layer itself can be about 0.01%, about 0.0125%, about 0.015%, about 0.0175%, about 0.02%, about 0.0225%, about 0.0125%, about 0.0275%, about 0.03%, about 0.0325%, about 0.035%, about 0.0375%, about 0.04%, about 0.0425%, about 0.045%, about 0.0475%, about 0.05%, about 0.0525%, about 0.055%, about 0.0575%, about 0.06%, about 0.0625%, about 0.065%, about 0.0675%, about 0.07%, about 0.0725%, about 0.075%, about 0.0775%, about 0.08%, about 0.0825%, about 0.085%, about 0.0875%, about 0.09%, about 0.0925%, about 0.095%, about 0.0975%, about 0.1%, about 0.125%, about 0.15%, about 0.175%, about 0.2%, about 0.225%, about 0.125%, about 0.275%, about 0.3%, about 0.325%, about 0.35%, about 0.375%, about 0.4%, about 0.425%, about 0.45%, about 0.475%, about 0.5%, about 0.525%, about 0.55%, about 0.575%, about 0.6%, about 0.625%, about 0.65%, about 0.675%, about 0.7%, about 0.725%, about 0.75%, about 0.775%, about 0.8%, about 0.825%, about 0.85%, about 0.875%, about 0.9%, about 0.925%, about 0.95%, about 0.975%, about 1%, about 1.125%, about 1.15%, about 1.175%, about 1.2%, about 1.225%, about 1.125%, about 1.275%, about 1.3%, about 1.325%, about 1.35%, about 1.375%, about 1.4%, about 1.425%, about 1.45%, about 1.475%, about 1.5%, about 1.525%, about 1.55%, about 1.575%, about 1.6%, about 1.625%, about 1.65%, about 1.675%, about 1.7%, about 1.725%, about 1.75%, about 1.775%, about 1.8%, about 1.825%, about 1.85%, about 1.875%, about 1.9%, about 1.925%, about 1.95%, about 1.975%, or about 2% (w/v, w/w or v/v). In some embodiments of any one of the aspects described herein, the amount of the conductive element in a composition used for preparing the polymeric coating layer or the polymeric coating layer itself is from about 0.01% to about 2%, about 0.05% to about 1.75%, about 0.1% to about 1.5%, about 0.35% to about 1%, or about 0.25% to about 0.75% (w/v, w/w or v/v). For example, the amount of the conductive element in the composition or the polymeric coating layer is about 0.5% (w/v, w/w or v/v).

### Redox Active Material

Embodiments of the various aspects described herein include a redox active material. As used herein, term “redox active material” refers to any chemical moiety capable of undergoing a reduction (accepting of an electron(s)) or oxidation (donation of an electron(s)) in the course of a multi-step process transferring electrons to or from a substrate of an oxidoreductase to an electrode. Exemplary redox active materials include, but are not limited to, metallocenes, metallocence derivatives, quinones, polycyclic aromatic hydrocarbons, redox-active organic molecules, phenazines, organosulfur compounds, radical acceptors, or a combination thereof.

In some embodiments of any one of the aspects described herein, the redox active material is selected from the group consisting of ferrocene, ferrocene derivatives, 3,3′,5,5′-tetramethylbenzidine (TMB), o-phenylenediamine dihydrochloride (OPD), N,N,N′,N′-tetramethyl-p-phenylenediamine, viologens 2,2′-azino-bis(3-ethylbenzothiazoline-6-sulfonic acid) (ABTS), p-nitrophenyl Phosphate (PNPP), 3,3′-diaminobenzidine (DAB), 4-chloro-1-naphthol (4-CN), 5-bromo-4-chloro-3-indolyl-phosphate (BCIP), nitro blue tetrazolium (NBT), methylene blue, tetrathialfulvaene, 2,6-dichloroindophenol (DCIP), 2,6-dichloroindophenyl phosphate, riboflavin 5′-monophosphate (RMP), ethyl viologen (1,1′-bis(ethyl)-4,4′-bipyridinium or BEBP), Paraquat, hexaamine ruthenium, 1,4-naphthoquione, resorufine, cyanomethylviologen, diquat, triquat, 1,1′-bis(2-sulfoethyl)-4,4′-bipyridinium, 1,1′-dibenzyl-4,4′-bipyridinium, 4,4′-dicarboxy-2,2′-bipyridyl, 1-hydroxybenzotriazole, veratryl alcohol, violuric acid, 2-methoxy-phenothiazone,3-hydroxyanthranilic acid, anthraquinone 2,6-disulfonic acid, N-hydroxyacetanilide, phenol red, 3,3′,5,5′-tetramethyl benzidine, dichlorophenol red, 2,2′,6,6′-tetramethylpiperidine-N-oxyl radical, syringaldehyde and acetosyringone, 2,2,6,6-tetramethylpiperidin-1-yl)oxyl (TEMPO), 2,2′7,7′-tetrakis-(N,N-di-p-methoxyphenyl-amine)-9,9′-spirobifluorene (spiro-MeOTAD), sodium anthraquinone-2,6-di sulphonate (AQDS), benzoquinones, 2,2′-biimidazole, 2-(2-pyridyl)imidazole, 2,2′-bipyridine, tetracyanoquinodimethane (TCNQ), and combinations thereof.

In some embodiments of any one of the aspects described herein, the redox active material is ferrocene or a derivative thereof. As used herein, the term “ferrocene derivative” refers to a molecule containing an optionally substituted ferrocene group (e.g., optionally substituted ferrocenyl radical or ferrocene nucleus). Some exemplary ferrocene derivatives include, but are not limited to, aminoferrocene, ferrocene monocarboxylic acid, ferrocyanide, 1,1′-ferrocene dicarboxylic acid, 1,1′-dimethylferrocene (DMF), polyvinylferrocene, [N-ferrocenoyl]-4-aminophenyl phosphate, ferrocenylmethyl methacrylate, β-ferrocenyl-propenoic acid, and ferrocene monocarboxylic acid (FMCA). In some embodiments of any one of the aspects described herein, the redox active material is aminoferrocene

In some embodiments of any one of the aspects described herein, the redox active material is covalently linked with other components present in the polymeric coating layer. For example, the redox active material can be covalently linked with the conductive element or the polymer in the in the polymeric coating layer. For example, the redox active material is covalently linked to the polymer in the in the polymeric coating layer. In another non-limiting example, the redox active material is covalently linked to the conductive element in the in the polymeric coating layer.

In some embodiments of any one of the aspects described herein, the redox active material in the polymeric coating layer is covalently linked to the substrate surface, e.g., conductive substrate surface coated with the polymeric coating layer. It is noted the redox active material can be linked directly to the surface without being linked to the conductive element or the polymer in the in the polymeric coating layer. In some embodiments, the redox active material is not linked directly to the surface. For example, the redox active material is linked to the surface by forming a covalent link to the conductive element or the polymer.

It is noted that the redox active material can be covalently linked with the conductive element, the polymer or the substrate surface directly (e.g., a bond) or by a cross-linking agent. Exemplary cross-linking agents include, but are not limited to, glutaraldehyde, Genipin, polyethylene glycol, carbodiimide based cross-linkers. Accordingly, in some embodiments, the redox active material is covalently linked with the conductive element by a cross-linking agent. For example, the redox active material covalently linked with the conductive element by a cross-linking agent selected from glutaraldehyde, Genipin, polyethylene glycol, and carbodiimide cross-linker. In some embodiments, the redox active material is covalently linked to the conductive element by formation of a bond between a functional group in the redox active material and a complementary functional group in the conductive element. In some other embodiments, the redox active material is covalently linked to the polymer by formation of a bond between a functional group in the redox active material and a complementary functional group in the polymer.

The ratio of the redox active material to the cross-linking agent can be from about 100:1 to about 1:1 (w/w). In some embodiments of any one of the aspects described herein, the ratio of the redox active material to the cross-linker is from about 100:1 to about 10:1 (w/w). For example, the ratio of the redox active material to the cross-linker can be from about 90:1 to about 20:1, about 80:1 to about 30:1, about 70:1 to about 40:1, or about 60:1 to about 50:1 (w/w). In some embodiments, the w/w ratio of the redox active material to cross-linker is about 100:1, or about 95:1, or about 90:1, or about 85:1, or about 80:1, about 75:1, or about 70:1, or about 65:1, or about 60:1, about 55:1, or about 50:1, or about 45:1, or about 40:1, about 35:1, or about 30:1, or about 25:1, or about 20:1, or about 15:1, or about 10:1, or about 5:1 or about 1:1.

The redox active material can be present in the polymeric coating layer in an amount from about 0.001 to about 10 (w/w).

The ratio of the redox active material to the polymer in the polymeric coating layer can be from about 1:10 to about 1:1000 (w/w). In some embodiments of any one of the aspects described herein, the ratio of the redox active material to the polymer in the polymeric coating layer is from about 1:10 to about 1:900 (w/w). For example, the ratio of the redox active material to the polymer in the polymeric coating layer can be from about 1:100 to about 1:800, about 1:200 to about 1:700, about 1:300 to about 1:600, or about 1:400 to about 1:500 (w/w). In some embodiments, the w/w ratio of the redox active material to the polymer in the polymeric coating is about 1:10, or about 1:50, or about 1:100, or about 1:150, or about 1:200, about 1:250, or about 1:300, or about 1:350, or about 1:400, about 1:450, or about 1:500, or about 1:550, or about 1:600, about 1:650, or about 1:700, or about 1:750, or about 1:800, or about 1:850, or about 1:900, or about 1:950, or about 1:1000.

The ratio of the redox active material to the conductive element in the polymeric coating layer can be from about 1:10 to about 1:1000 (w/w). In some embodiments of any one of the aspects described herein, the ratio of the redox active material to the conductive element in the polymeric coating layer is from about 1:10 to about 1:900 (w/w). For example, the ratio of the redox active material to the conductive element in the polymeric coating layer can be from about 1:100 to about 1:800, about 1:200 to about 1:700, about 1:300 to about 1:600, or about 1:400 to about 1:500 (w/w). In some embodiments, the w/w ratio of the redox active material to the conductive element in the polymeric coating is about 1:10, or about 1:50, or about 1:100, or about 1:150, or about 1:200, about 1:250, or about 1:300, or about 1:350, or about 1:400, about 1:450, or about 1:500, or about 1:550, or about 1:600, about 1:650, or about 1:700, or about 1:750, or about 1:800, or about 1:850, or about 1:900, or about 1:950, or about 1:1000.

### Electrode

As used herein an “electrode” is a conductor through which current enters or leaves a medium, where the medium is nonmetallic (i.e., it emits or collects electrons or electron “holes”). For example, the medium can be a complex matrix (e.g., blood or serum). The electrode can be inserted into/onto a tissue such as mammalian tissue and be contacted with tissue and/or fluids therein/thereon. The electrode can be large (e.g., with a working surface area of greater than 1 cm², greater than 10 cm², greater than 100 cm²) or the electrode can be small (e.g., with a working surface area of less than 1 cm², less than 1 mm², less than 100 μm², less than 10 μm², less than 1 μm²). The working surface area is the area in contact with the medium and wherein current enters or leaves the medium.

The conductive substrate can be in any form having a surface that can be coated. For example, the conductive substrate can be included in the form of a conductive particle, a conductive nano-particle, a conductive micro-particle, a conductive nano-fiber, a conductive micro-fiber, a conductive flake, a conductive chip, a conductive crystal, a conductive porous substrate, a conductive wafer, a conductive wire, a conductive nano-wire, a conductive micro-wire, a conductive channel, a conductive nano-channel, a conductive micro-channel, a conductive rod, a conductive nano-rod, a conductive micro-rod, a conductive foil, a conductive sheet, a conductive web, or combination of these forms. In some implementations, the conductive substrate is part of a microfluidic device, such as a channel or chamber therein.

Metal patterning techniques, such as standard printed circuit board (PCB) technology, offer a number of versatile fabrication options such as (i) track size and spacing less than 100 μm; (ii) high purity electrolytic gold plating several microns thick suitable for electrochemistry and surface modification chemistries; (iii) ease of small-scale prototyping in standard laboratory settings; and (iv) large scale mass manufacturing capabilities at a fraction of the cost of high-end microarrays. In some embodiments, electrodes as disclosed herein may be fabricated using PCB technology.

In some embodiments, the electrodes are mass fabricated onto non-electrically conductive surfaces such as plastic substrates using inexpensive standard technology such as printed circuit board (PCB) technology, roll-to-roll laser ablation or evaporation. Exemplary non-electrically conductive surfaces include plastic, poly(carbonate) (PC), poly(methyl methacrylate) (PMMA), cyclic olefin polymers (COP) or cyclic olefin copolymers (COC), SU-8, parylene, silicon nitride, kapton, styrene-ethylene-butylene-styrene (SEBS), poly-dimethylsiloxane (PDMS), polyimide, silicon dioxide, and any combination thereof.

In some embodiments, the electrode is a planar or a 3-dimensional electrode. As used herein, a planar electrode electrically interacts with an electroactive species or mediator on a 2-dimensional surface. As used herein, a 3-dimensional electrode is an electrode displaying a very high surface area per unit volume, caused by no planarity. Without being bound by theory, this provides high turbulence at their interface with an electroactive species or mediator, enhancing the mass transfer process of the electroactive species towards the electrode surface. These characteristics strongly improve the electrochemical reaction rate.

In some embodiments the electrode is “Multiplexed” such that it is configured for a multiplexed assay. As used herein a “multiplexed” assay can be used to simultaneously measure multiple analytes or signals such as two or more (e.g., 3 or more, 5 or more, 10 or more, 50 or more, 100 or more, 1000 or more) during a single run or cycle of the assay. The electrode can therefore be configured as an array of electrodes, microelectrodes or electrochemical sensors each of which can be independently electrically attached to a circuit for monitoring the electrical signals. For example, the array of electrodes can be disposed at the bottom, sides or top of a multiwell plate (e.g., microwell plate) arrayed on a flat surface such as a semiconductor chip (e.g., a sensor array chip) or form part of a multielectrode array (e.g., for connection of neurons to electronic circuitry). In some embodiments, the compositions as described herein, can coat more than one sensor since the coating will not conduct between the sensors due to the anisotropy of the conduction, therefore an array of conductors, sensors or electrodes can be coated forming a multiplexed electrode.

Electrodes can include materials with metallic conduction and semiconductors. For example, electrodes can include metals, metal alloys, semiconductors, doped materials, conducting ceramics and conducting polymers. Without limitation, electrode materials can include carbon (e.g., graphite, glassy carbon, conductive polymers), copper, titanium, brass, mercury, silver, platinum, palladium, gold, rhodium, zinc, lead, tin, iron, Indium Tin Oxide (ITO), aluminum, stainless steel, tungsten, nickel, zirconium, niobium, tantalum, chromium, molybdenum, manganese, rhenium, ruthenium, rhodium, iridium, silicon, doped silicon, II-VI semiconductors (e.g., ZnO, ZnS, CdSe), III-V semiconductors such as (e, g., GaAs, InSb), ceramics (e.g., TiO₂, Fe₃O₄, MgCr₂O₄), and conductive polymers (e.g., poly(acetylene)s, poly(p-phenylene vinylene), poly(fluorenes)s, polyphenylenes, polypyrenes, polyazulenes, polynaphthalenes, polyanilines, polyazepines, polyindoles, polycarbazoles, poly(pyrrole)s, poly(thiophene)s, poly(3,4-ethylenedioxythiophene)), polyimide, parylene, benzocyclobutene, and combinations, mixtures and alloys of these.

In some embodiments, the conductive substrate includes a metal, a metalloid, a conducting polymer, a conducting glassy material, a conducting amorphous material, a conducting biological membrane, a conducting carbon-based material, or any combination of these.

In some embodiments, the conductive substrate includes gold. In some embodiments, the conductive substrate includes a silica-based glass (e.g., pure silica or mixtures such as borosilicate glass). In some embodiments, the conductive substrate includes graphite, diamond, glassy carbon, or carbon nano-tubes (CNTs). In some implementations, the conductive substrate is a chip including gold and a silica-based glass.

In some embodiments of any one of the aspects described herein, the conductive substrate is a flexible substrate. For example, the conductive substrate comprises a flexible material. Exemplary materials for the flexible substrate include, but are not limited to, polyethylene terephthalate, polyethylene naphathalate, polyimides, polymeric hydrocarbons, celluloses, plastics, polycarbonates, polystyrenes, and any combination thereof.

Electrodes can also include insulating components such as insulators for electrical and mechanical protection, imparting rigidity and electrical isolation to parts of the electrode.

In some embodiments, the electrode can be part of an electrochemical cell. For example, the electrode is a working electrode and the electrochemical cell can include a counter electrode and reference electrode.

Electrochemical methods are methods that rely on a change in the potential, charge or current to characterize the analyte's chemical reactivity. Some examples include potentiometry, controlled current coulometry, controlled-potential coulometry, amperometry, stripping voltammetry, hydrodynamic voltammetry, polarography, stationary electrode voltammetry, pulsed polarography, electrochemical impedance spectroscopy and cyclic voltammetry. The signals are detected using an electrode or electrochemical sensors coupled to circuits and systems for collection, manipulation and analysis of the signals.

In some embodiments, the polymeric coating layer of the electrode is adapted for contact with an analyte or a sample comprising an analyte. The polymeric coating layer can allow analyte to flow through the pores and be detected, for example, by binding to a capture molecule such as an antibody, DNA strand, or aptamer. In some implementations, the coatings can be patterned as a conductive wire or a dielectric/insulating surface. Some implementations include coating microfluidic chips, lab-on-a-chip, and organs on a chip. In some implementations, the coatings can be used in nano-gap and micro-gap devices. For example, these devices include nano-gap electrodes, nanostructured-based electrical biosensors, and nano-gap dielectric biosensor for label-free DNA hybridization detection. The coatings can be applied, for example, to the gap between electrodes in the device.

### Sensors

In another aspect provided herein is a sensor comprising an electrode as described herein. In the context of this specification, the term “sensor” refers a device that senses the presence and/or amount of something. For example, the sensor could sense the presence of a chemical such as glucose, a protein such as an antigen, or an antibody in a biological fluid.

A sensor has two basic components: the sensing surface (or receptor) and the transducer. The sensing surface interacts with the target analyte and the transducer converts this interaction into a readable electronic signal. The sensor performance characteristics depend on both the components. The sensor selectivity and affinity towards the target analyte depends solely on the sensing surface because the analyte interacts only at the sensing surface. Other performance metrics such as sensitivity, resolution, and calibration depend on both components.

The sensor may have a channel length of between about 5 μm and about 50 μm, or between about 10 μm and about 30 μm, or about 20 μm, and a channel width of between about 1 mm and about 20 mm, or between about 1 mm and about 10 mm, or about 3 mm.

In some embodiments, the sensor has one or more fluid-contact surfaces, and the electrode is immobilized on at least a portion of the fluid contact surface. In some embodiments, the sensor has one or more wells. In some embodiments, each well of the sensor comprises an inner bottom surface on which one or more analyte specific electrodes are immobilized. In some embodiments, the wells are open cells comprising open tops, enclosed sides and bottom, and one or more analyte-specific electrodes immobilized on the inner fluid-contact surface of the wells. In some embodiments, the sensor comprises 1, 2, 3, 4, 5, 6, 8, 10, 12, 16, 24, 32, 48, 64, 96 or more open wells. In one embodiment the sensor is in the form of a 96-well microtiter plate.

In some embodiments, the wells are microfluidic flow cells comprising an enclosed top, sides and bottom, wherein the top of each flow cell includes a fluid inlet and a fluid outlet, and comprising one or more analyte-specific electrodes immobilized on the inner fluid-contact surface of the wells. In some embodiments, the electrochemical sensor comprises 1, 2, 3, 4, 5, 6, 8, 10, 12, 16, 24, 32, 48, 64, 96 or more microfluidic flow cells. Another embodiment is in the form of a 96-well microtiter plate, wherein each well comprises an enclosed top having a fluid inlet and a fluid outlet. In some embodiments, the sensor comprises both one or more open cells and one or more flow cells. Each well contains an array of analyte-specific electrodes (e.g., 32 gold electrodes) that can be individually modified with capture probes to bind the corresponding target analyte (e.g., pathogen, protein, carbohydrate, toxin, drug, etc.) present in the collected sample. In some embodiments, one sample is introduced into each well. In embodiments having two or more wells, portions of the same sample can be introduced into more than one well, or different samples can be introduced into different wells. Thus, in embodiments having multiple wells, multiple samples can be simultaneously assayed.

Also provided herein is the use of a sensor. In some embodiments, the sensor may be for sensing an analyte in a sample. The analyte is optionally a biological analyte. In an embodiment the analyte is an antibody, antigen, protein, peptide or chemical. The sample may be any aqueous solution but is preferably a biological fluid, more preferably a bodily fluid, and still more preferably, saliva.

### Analyte Detection

The electrode and sensors described herein can be used for detecting analytes, e.g., in a sample. Accordingly, another aspect provided herein relates to methods of detecting at least one target analyte, including, e.g., at least 2, 3, 4, 5, 6, 7, 8 target analytes or more. Generally, the method comprises contacting a sample suspected of comprising a target analyte with an electrode or sensor described herein and detecting the binding of the target analyte with the target binding ligand. The binding may be detected electrochemically.

In some embodiments, the method of detecting a target analyte comprises contacting a sample suspected of comprising a target analyte with an electrode or sensor described herein and detecting the binding of the target analyte with the target binding ligand. Optionally, detecting the binding of the target molecule with the target binding ligand comprises applying a voltage to the electrode and measuring the current generated from the electrode.

In some embodiments of any one of the aspects described herein, the electrode or the sensor is configured for label-free detection. As used herein, the term “label-free” describes a detection method wherein the detectability of an analyte is not dependent upon the presence or absence of a detectable label. In other words, no further signal enhancers are required in order to detect the binding of the target analyte with the target-binding molecule.

In some other embodiments, the electrode or the sensor is configured for detection of a label.

The applied voltage provides a sufficiently strong electric field to liberate H⁺ from H₂O₂, but not strong enough to cause electrolysis of water, as electrolysis of water may lead to a decrease in the signal-to-noise ratio (i.e. below −1.23 V). Typically, the voltage applied is between about 0 V and −2 V, e.g. about −1 V. In some embodiments, low voltages can be used including 250 to 500 m V to 300-400 mV. The voltage range can then be −0.25to −2 V or −0.25 to −0.1 V.

In some embodiments, the target of the target binding molecule can be redox active (e.g., an electroactive analyte) and is directly detected by an electrode. For example, the target binding molecule facilitates detection of the target analyte by the electrode due to it concentrating the analyte near or at the surface of the electrode where it can be detected directly by electrochemical means.

In some embodiments, the binding of the target analyte to the target binding molecule is detected indirectly by electrochemical means. For example, the target can be detected by binding with a detection agent that catalyzes, directly or indirectly, a redox reaction close to an electrode surface. For example, the target analyte can be contacted with a labeling probe, e.g., a second target binding molecule, wherein the labeling probe comprises a detectable label. Generally, the labeling probe comprises a target binding molecule capable of binding with the target analyte. For example, the labeling probe is a target binding molecule described herein. In some embodiments, the labeling probe is an antibody, antigen binding fragment of an antibody, an antigen, a receptor, a ligand for a receptor, an enzyme, or a nucleic acid.

As used herein, the term “detectable label” refers to a molecule or composition capable of producing a detectable signal indicative of the presence of a target. Exemplary detectable labels include but are not limited to an enzyme, a fluorophore, a chemiluminescent label, colloidal gold, colored glass or plastic (e.g., polystyrene, polypropylene, and latex) beads, a radiolabel, a quantum dot, or any combination thereof.

In some embodiments, the labeling probe contacts the target analyte prior to contacting the sample with the electrode or. In some other embodiments, the labeling probe contacts the target analyte after contacting the sample with the electrode or. In some embodiments of any one of the aspects described herein, the detectable label deposits a sacrificial redox active molecule on the electrode surface (e.g., on a coating that is on the surface of the electrode) that then is detected electrochemically.

In some embodiments of any one of the aspects described herein, the detectable label comprises an enzyme. Non-limiting examples of enzymes include: a peroxidase, alkaline phosphatase, malate dehydrogenase, staphylococcal nuclease, delta-V-steroid isomerase, yeast alcohol dehydrogenase, alpha-glycerophosphate dehydrogenase, triose phosphate isomerase, horseradish peroxidase (HRP), alkaline phosphatase, asparaginase, glucose oxidase, beta-galactosidase, ribonuclease, urease, catalase, glucose-VI-phosphate dehydrogenase, glucoamylase, tyrosinase, acetylcholinesterase, or any combination thereof. In some preferred embodiments, the enzyme is a peroxidase or alkaline phosphatase.

When the detectable label comprises an enzyme, the method can further comprise contacting the enzyme with a substrate of the enzyme. Exemplary reporter enzyme substrates include, but are not limited to, hydrogen peroxide, carbamide peroxide, nucleotides, oligonucleotides, RNA, DNA, phosphorylated peptides, phosphorylated proteins, phosphorylated small molecules, glucose, phenols, tyrosine, dopamine, catechol, urea, and any combination thereof.

In some embodiments, the reporter enzyme substrate is hydrogen peroxide. In some embodiments the target analyte can be redox active and the enzyme is directly responsible for generation of a charge carrier that is detected by an electrode. For example, the binding of the target analyte to the second target binding molecule facilitates generation of the charge carrier near the polymeric coating layer surface, the conducting polymeric coating layer conducts the charge carrier and this impacts the applied voltage and/or current resulting in detection of the target analyte.

The charge carrier can be any one or more of the following charge carrier types: anions, cations or electrons. In some embodiments, the charge carriers are cations, e.g., hydrogen ions such as protons).

In some embodiments, the substrate of the enzyme can be redox active and the enzyme is directly responsible for generation of a charge carrier that is detected by an electrode. For example, the enzyme facilitates generation of the charge carrier near the polymeric coating layer surface, the polymeric layer conducts the charge carrier and this impacts the applied voltage and/or current resulting in detection of the target analyte.

In some embodiments, the enzyme is a redox catalyst and, in the presence of the substrate for said enzyme, the substrate for the reported enzyme is oxidized or reduced, thereby generating a charge carrier. Non-limiting examples of redox active molecules that can be oxidized or reduced and can be substrates to a redox catalyst include, 3,3′,5,5′-tetramethylbenzidine (TMB), o-phenylenediamine dihydrochloride (OPD), 2,2′-Azinobis [3-ethylbenzothiazoline-6-sulfonic acid] (ABTS), p-Nitrophenyl Phosphate (PNPP), 3,3′-diaminobenzidine (DAB), 4-chloro-1-naphthol (4-CN), 5-bromo-4-chloro-3-indolyl-phosphate (BCIP), nitro blue tetrazolium (NBT), methylene blue, hydroquinone, ferrocene derivatives, and any combination thereof.

In some embodiments of any one of the aspects, the binding of the target analyte to the target binding molecule can be detected using the methods described in U.S. Ser. No. 10/753,940, content of which is incorporated herein by reference in its entirety. For example, where the detectable label comprises an enzyme, the method comprises contacting the labeling probe, e.g., labeling probe bound to the target analyte with a reporter enzyme substrate, an electroactive mediator and, optionally, a precipitating agent. It is noted the reporter enzyme substrate, the electroactive mediator and, optionally, the precipitating agent can be contacted with the labeling probe simultaneously or serially. In some embodiments, the reporter enzyme substrate, the electroactive mediator and, optionally, the precipitating agent are contacted simultaneously with the labeling probe. Without wishing to be bound by a reaction the reporter enzyme substrate, the electroactive mediator and, optionally, the precipitating agent with the enzyme conjugated with the label probe forms an electroactive precipitate which is locally deposited near or at the surface of the electrode.

Exemplary electroactive mediators include, but are not limited to, 3,3′,5,5′-tetramethylbenzidine (TMB), o-phenylenediamine dihydrochloride (OPD), 2,2′-Azinobis [3-ethylbenzothiazoline-6-sulfonic acid] (ABTS), p-Nitrophenyl Phosphate (PNPP), 3,3′-diaminobenzidine (DAB), 4-chloro-1-naphthol (4-CN), 5-bromo-4-chloro-3-indolyl-phosphate (BCIP), nitro blue tetrazolium (NBT), methylene blue, hydroquinone, ferrocene derivatives, and any combination thereof. In some embodiments, the electroactive mediator is TMB.

Exemplary precipitating agents include, but are not limited to, a water-soluble polymer, a pyrrolidinone polymer, a polyaniline, a polypyrrole, a polythiophene, alginic acid, methyl vinyl ether/maleic anhydride copolymer, dextran sulfate, carrageenan, and any combination thereof. In some embodiments, the precipitating agent is a pyrrolidinone polymer.

In some embodiments of any one of the aspects described herein, the reporter enzyme substrate, the electroactive mediator and, optionally, the precipitating agent are comprised in a composition for contacting with the labeling probe.

In some embodiments, the voltage applied corresponds to an electrochemical oxidation or reduction potential, or combination thereof, of the electroactive mediator in a fully or partially oxidized state. In some embodiments, the generated current corresponds to a reduction or oxidation current derived from reduction or oxidation of the fully or partially oxidized electroactive mediator. An exemplary voltage window includes, but is not limited to, about −0.2V as reduction potential to +0.2V as oxidation potential versus a reference electrode In some implementations, the capture agent is used at a concentration between about 10 and about 5000 μg/mL. In some implementations, the capture agent is used at a concentration between about 50 and 1000 μ/mL, such as between about 100 and 1000 μ/mL, or between about 100 and about 1000 μ/mL. In some implementations, the labeling probe is used at a concentration between about 0.1 and 100 μ/mL, such as between about 0.5 and 50 μ/mL, between about 1 and 20 μ/mL, between about 1 and 8 μ/mL, or between about 2 and 5 μ/mL. In some implementations, the labeling probe includes streptavidin-polyHRP or a similar molecule for signal augmentation. In some implementations, the streptavidin-polyHRP concentration is between about 0.1 and about 100 μ/mL, such as between about 0.5 and 50 μ/mL, or between about 1 and 10 μ/mL. The ranges of concentrations of capture agent and labeling probe can be used in any combination, such as 500 μ/ml of capture agent in combination with 5 μ/ml of labeling probe. The ranges of concentrations of capture agent, labeling probe and streptavidin-polyHRP also be used in any combination, such as 500 μ/mL of capture agent, 5 μ/mL of labeling probe and 2 μ/mL streptavidin-polyHRP.

### Target Analytes

In some embodiments, the analyte is a biological analyte. In some embodiments, the analyte ion, molecule, oligomer, polymer, protein, peptide, polypeptide, peptidomimetic, nucleic acid, antigen, antibody, nucleic acid, toxin, biological threat agent such as spore, viral, cellular and protein toxin, carbohydrate, monosaccharide, disaccharide, oligosaccharide, polyol, and polysaccharide, lipid, peptidoglycan, cell, microbial matter, steroid, hormone, lipopolysaccharide, endotoxin, therapeutic agent, lipid-binding molecule, co-factor, small molecule, fatty acid, chemical, or combinations of these. The analyte is optionally an antigen or antibody indicative of infection or resistance to infection. The analyte is optionally a clinical chemistry analyte.

In some embodiments, the analyte is immunological or serological, for example an antigen or antibody.

In some embodiments the analyte is a hormone, for example a gynaecological hormone such as luteinizing hormone (LH), progesterone, estradiol or follicle-stimulating hormone. In preferred embodiments the probe detects LH. In some embodiments the probe is a LH specific antibody. In some embodiments the probe is an LH monoclonal antibody. Additionally, or alternatively the hormone may be a pregnancy hormone such as human chorionic gonadotropin (hCG).

In some embodiments the analyte is a clinical chemistry analyte such as an ion, salt, mineral, metabolite, therapeutic drug, toxicology marker, drug of abuse, transport protein, enzyme, specific protein, lipoprotein or marker, for example diabetes or myocardial infarction markers. In some embodiments the analyte is a metabolite selected from the group of glucose, cholesterol, urea, lactic acid, bilirubin, creatinine, triglycerides. In preferred embodiments the probe is selected to detect glucose or cholesterol.

In some embodiments, the analyte is a tumor marker. Tumor markers can be used in guiding treatment decisions, monitoring treatment, predicting the change of recovery and to predict or monitor for tumor recurrence.

### Sample

In accordance with various embodiments described herein, a sample, including any fluid or specimen (processed or unprocessed) that is intended to be evaluated for the presence of an analyte can be subjected to methods, compositions, kits and systems described herein. The sample or fluid can be liquid, supercritical fluid, solutions, suspensions, gases, gels, slurries, and combinations thereof. The sample or fluid can be aqueous or non-aqueous.

In some embodiments, the sample can be an aqueous fluid. An aqueous fluid includes biological fluids as described below. Optionally, if the sample is water-based but not fluid, an aqueous solution can be added to produce a fluid sample.

In some embodiments, the sample can include a biological fluid obtained from a subject. Exemplary biological fluids obtained from a subject can include, but are not limited to, blood (including whole blood, plasma, cord blood and serum), lactation products (e.g., milk), amniotic fluids, sputum, saliva, urine, semen, cerebrospinal fluid, bronchial aspirate, perspiration, mucus, liquefied stool sample, synovial fluid, lymphatic fluid, tears, tracheal aspirate, breath condensate and any combination thereof. In some embodiments, a biological fluid can include a homogenate of a tissue specimen (e.g., biopsy) from a subject. In one embodiment, a test sample can comprise a suspension obtained from homogenization of a solid sample or a fragment thereof obtained from a subject.

In some embodiments, the sample can include a fluid or specimen obtained from an environmental source. For example, the fluid or specimen obtained from the environmental source can be obtained or derived from food products or industrial food products, food produce, poultry, meat, fish, beverages, grains, crops, dairy products, water (including wastewater), surfaces, ponds, rivers, reservoirs, swimming pools, soils, food processing and/or packaging plants, agricultural places, hydrocultures (including hydroponic food farms), pharmaceutical manufacturing plants, animal colony facilities, and any combinations thereof.

In some embodiments, the sample can be a non-biological fluid. As used herein, the term “non-biological fluid” refers to any fluid that is not a biological fluid as the term is defined herein. Exemplary non-biological fluids include, but are not limited to, water, salt water, brine, drinking water, industrial water, brown water, sewerage, and mixtures thereof. Preferred non-biological fluids are drinking or industrial water or sewerage.

In some embodiments, the sample is pre-processed prior to contacting with the electrode or the sensor.

### Linkers

Embodiments of the various aspects described herein include a linker. For example, the target binding molecule can be linked to the surface of the conductive substrate via a linker. As used herein, the term “linker” means an organic moiety that connects two parts of a compound. Linkers typically comprise a direct bond or an atom such as oxygen or sulfur, a unit such as NR¹, C(O), C(O)O, C(O)NR¹, SO, SO₂, SO₂NH or a chain of atoms, such as substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl,, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, where one or more methylenes can be interrupted or terminated by O, S, S(O), SO₂, N(R^{LL})₂, C(O), cleavable linking group, substituted or unsubstituted aryl, substituted or unsubstituted heteroaryl, substituted or unsubstituted heterocyclic; where R¹ and R^{LL} independently are is hydrogen, acyl, aliphatic or substituted aliphatic.

In some embodiments of any of the aspects, the linker is a flexible linker. As used herein, a “flexible linker” is a linker which does not have a fixed structure (secondary or tertiary structure) in solution and is therefore free to adopt a variety of conformations. Generally, a flexible linker has a plurality of freely rotating bonds along its backbone. In contrast, a rigid linker is a linker which adopts a relatively well-defined conformation when in solution. Rigid linkers are therefore those which have a particular secondary and/or tertiary structure in solution. In some embodiment, the linker is a rigid linker.

### Kits

In another aspect, the present disclosure provides a kit comprising a surface, electrode, or sensor described herein.

In addition to the above-mentioned components, any embodiments of the kits described herein can include informational material. The informational material can be descriptive, instructional, marketing or other material that relates to the methods described herein and/or the use of the aggregates for the methods described herein. For example, the informational material can describe methods for using the kits provided herein to perform an assay for capture and/or detection of a target analyte. The kit can also include an empty container and/or a delivery device, e.g., which can be used to deliver a test sample to a test container.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is a link or contact information, e.g., a physical address, email address, hyperlink, website, or telephone number, where a user of the kit can obtain substantive information about the formulation and/or its use in the methods described herein. Of course, the informational material can also be provided in any combination of formats.

In some embodiments, the kit can contain separate containers, dividers or compartments for each component and informational material. For example, each different component can be contained in a bottle, vial, or syringe, and the informational material can be contained in a plastic sleeve or packet. In other embodiments, the separate elements of the kit are contained within a single, undivided container.

Some exemplary aspects of the disclosure are described by one or more of following numbered Embodiments:

Embodiment 1: An electrode comprising: (i) a conductive substrate; (ii) a target-binding molecule immobilized on surface of the conductive substrate, wherein the target-binding molecule is capable of binding with a target molecule; and (iii) and a polymeric coating layer comprising a polymer on said surface of the conductive substrate, and wherein the polymeric coating layer covers at least a non-target binding portion of the target-binding molecule.

Embodiment 2: The electrode of Embodiment 1, wherein an electric double layer (EDL) is closer to a target-binding portion or site of the target-binding molecule than the surface of the conductive substrate.

Embodiment 3: The electrode of Embodiment 1 or 2, wherein the polymeric coating layer forms an insulating or non-conducting layer between a target-binding portion or site of the target-binding molecule and the surface of the conductive substrate.

Embodiment 4: The electrode of any one of Embodiments 1-3, wherein a target-binding portion or site of the target-binding molecule is not embedded within the polymeric coating layer.

Embodiment 5: The electrode of any one of Embodiments 1-3, wherein a target-binding portion or site of the target-binding molecule is exposed for contact with an analyte, and wherein said contact with the analyte is not within the polymeric coating layer.

Embodiment 6: The electrode of any one of Embodiments 1-3, wherein a target-binding portion or site of the target-binding molecule is at a surface of the polymeric coating layer.

Embodiment 7: The electrode of any one of Embodiments 1-3, wherein the polymeric coating layer does not cover a target-binding portion or site of the target-binding molecule.

Embodiment 8: The electrode of any one of Embodiments 1-7, wherein a target-binding portion or site of the target-binding molecule is at a surface of the polymeric coating layer, and wherein said surface of the polymeric coating layer is not a surface of a pore in the polymeric coating layer.

Embodiment 9: The electrode of any one of Embodiments 1-8, wherein the polymeric coating layer reduces or inhibits mobility ions in a liquid solution to the surface of the conductive substrate when the electrode is disposed in said liquid solution.

Embodiment 10: The electrode of any one of Embodiments 1-9, wherein the polymeric coating layer reduces or inhibits direct contact between ions in a liquid solution and the surface of the conductive substrate when the electrode is disposed in said liquid solution.

Embodiment 11: The electrode of any one of Embodiments 1-10, wherein the polymeric coating layer has a porosity of about 10% (e.g., about 9%, about 8%, about 7%, about 6%, about 5%, about 4%, about 3% about 2.5%, about 2%, about 1.5%, about 1%, about 0.75%, about 0.5%) or lower.

Embodiment 12: The electrode of any one of Embodiments 1-11, wherein the polymeric coating layer is substantially non-porous.

Embodiment 13: The electrode of any one of Embodiments 1-10, wherein the polymeric coating layer is porous.

Embodiment 14: The electrode of Embodiment 13, wherein the polymeric coating layer has a porosity of about 5% to about 95%.

Embodiment 15: The electrode of any one of Embodiments 13-14, wherein the polymeric coating layer comprises macropores (e.g., pores having a diameter of from about 0.1 μm to about 10 μm such as from about 0.5 μm to about 5 μm or from about 1 μm to about 3 μm).

Embodiment 16: The electrode of any one of Embodiments 13-15, wherein the polymeric coating layer comprises mesopores (e.g., pores having a diameter from about 5 nm to about 99 nm).

Embodiment 17: The electrode of any one of Embodiments 13-16, wherein the polymeric coating layer comprises nanopores (e.g., pores having a diameter of from about 0.1 nm to about 4.5 nm).

Embodiment 18: The electrode of any one of Embodiments 1-17, wherein the polymeric coating layer completely covers the surface of the conductive substrate.

Embodiment 19: The electrode of any one of Embodiments 1-18, wherein a thickness of the polymeric coating layer is about 95% or less of a distance between a target-binding portion or site of the target-binding molecule and the surface of the conductive substrate.

Embodiment 20: The electrode of any one of Embodiments 1-19, wherein a thickness of the polymeric coating layer is at least about 1 nm.

Embodiment 21: The electrode of any one of Embodiments 1-20, wherein a thickness of the polymeric coating layer is about 20 nm or lower.

Embodiment 22: The electrode of any one of Embodiments 1-21, wherein the polymeric coating layer comprises an electropolymerized polymer, photopolymerized polymer, or auto-polymerized polymer.

Embodiment 23: The electrode of any one of Embodiment 1-22, wherein the polymer coating layer comprises a electropolymerized polymer.

Embodiment 24: The electrode of any one of Embodiment 1-23, wherein the polymeric coating layer comprises a polymer selected from the group consisting of polysaccharides, polydopamines, poly-p-phenylenes, polypyrroles, coumarin based polymers, polyhydroxyethylmethacrylates, polyindolyenes, polyanilines, polythiophenes, poly(ethylenedioxy) thiophene, poly(heteroaromatic vinylenes), polyvinylphosphates, poly-p-phenylene sulfides, poly(2,5-thienylenes), poly-o-phenylenes, poly(o-phenylene diamines), polysaccharides, polyacetylenes, porphyrins, phthalocyanins, fullerenes, polyparaphenylenes, polyphenylenevinylenes, polyfluorenes, polypyridines, polycarbazoles, polypyridinevinylenes, polyarylvinylenes, poly (p-phenylmethylvinylenes), derivatives and co-polymers thereof, and any combination thereof.

Embodiment 25: The electrode of any one of Embodiments 1-24, wherein the polymeric coating layer comprises polydopamine, chitosan, or scopoletin (6-methoxy-7-hydroxycoumarin).

Embodiment 26: The electrode of any one of Embodiment 1-25, wherein the polymeric coating layer comprises electropolymerized polydopamine, electropolymerized chitosan, electropolymerized scopoletin, or autopolymerized chitosan.

Embodiment 27: The electrode of any one of Embodiment 1-26, wherein the polymeric coating layer further comprises a dissolvable or degradable material.

Embodiment 28: The electrode of Embodiment 27, wherein the dissolvable or degradable material is selected from the group consisting of water dissolvable polymers (salt crystals), materials capable of being removed by degradation (e.g., proteins or protein aggregates), temperature dependent polymers (e.g., poly(N-isopropyl acrylamide) (PNIPAAm)); and any combination thereof.

Embodiment 29: The electrode of any one of Embodiments 1-28, wherein the polymeric coating layer further comprises an antifouling material.

Embodiment 30: The method of Embodiment 29, wherein the antifouling material is ethanolamine (ETA), hyaluronic acid (HA), or poly vinyl alcohol (PVA).

Embodiment 31: The electrode of any one of Embodiment 1-30, wherein the target-binding molecule is a peptide, polypeptide, a nucleic acid (e.g., oligonucleotide or polynucleotide) or a small molecule.

Embodiment 32: The electrode of any one of Embodiment 1-31, wherein the target binding molecule is a receptor, a ligand for a receptor, an antibody, antigen binding fragment of an antibody, an antigen, an enzyme, an affimer or an aptamer.

Embodiment 33: The electrode of one of Embodiments 1-32, wherein the target binding molecule is an antibody, an antigen binding fragment of an antibody, or an antigen, optionally, the target binding molecule is an antibody or an antigen binding fragment of an antibody.

Embodiment 34: The electrode of any one of Embodiments 1-33, wherein the electrode is a planar or 3-dimensional electrode.

Embodiment 35: The electrode of any one of Embodiments 1-34, wherein the conductive substrate comprises gold, silver, copper, platinum, aluminum, stainless steel, tungsten, indium tin oxide, titanium, lead, nickel, palladium, zirconium, niobium, tantalum, chromium, molybdenum, manganese, rhenium, ruthenium, rhodium, iridium, silicon, polyimide, parylene, benzocyclobutene, carbon, graphite, or any combination thereof.

Embodiment 36: The electrode of any one of Embodiments 1-35, wherein the conductive substrate comprises a flexible substrate.

Embodiment 37: The electrode of any one of Embodiments 1-36, wherein the conductive substrate comprises a flexible substrate selected from the group consisting of polyethylene terephthalate, polyethylene naphathalate, polyimides, polymeric hydrocarbons, celluloses, plastics, polycarbonates, polystyrenes, silicon and any combination thereof.

Embodiment 38: The electrode of any one of Embodiments 1-37, wherein the polymeric coating layer is adapted for contact with an analyte or a sample comprising an analyte.

Embodiment 39: The electrode of any one of Embodiments 1-38, wherein the polymeric coating layer further comprises a conducting material.

Embodiment 40: The electrode of Embodiment 39, wherein the conducting material comprises conductive particles, conductive rods, conductive fibers, conductive nano-particles, conductive polymers, conductive nano-flakes, conductive nanotubes, semi-conductive particles, semi-conductive rods, semi-conductive fibers, semi-conductive nano-particles, semi-conductive nanoflakes, semi-conductive nanotubes, or semi-conductive polymers.

Embodiment 41: The electrode of Embodiment 39 or 40, wherein the conducting material is a metal, a metalloid, conducting polymer, a conducting carbon-based material, organic compounds having conducting and/or semiconducting properties, or any combination thereof.

Embodiment 42: The electrode of any one of Embodiments 39-41, wherein the conducting material comprises an allotrope of carbon atoms arranged in a hexagonal lattice.

Embodiment 43: The electrode of any one of Embodiments 39-42, wherein the conducting material is graphite, functionalised graphene oxide, reduced graphene oxide, or carbon nano-tubes (CNTs).

Embodiment 44: The electrode of Embodiment 43, wherein the carbon nanotubes are carboxylated carbon nanotubes or aminated carbon nanotubes.

Embodiment 45: The electrode of Embodiment 43, wherein the reduced graphene oxide is a carboxylated reduced graphene oxide or an aminated reduced graphene oxide.

Embodiment 46: The electrode of any one of Embodiments 39-41, wherein the conductive material comprises gold.

Embodiment 47: The electrode of any one of Embodiments 1-41, wherein the polymeric coating layer further comprises a redox active material.

Embodiment 48: The electrode of Embodiment 47, wherein the redox active material is selected from the group consisting of metallocenes metallocence derivatives, quinones, polycyclic aromatic hydrocarbons, redox-active organic molecules, phenazines, organosulfur compounds, and radical acceptors.

Embodiment 49: The electrode of any one of Embodiments 47 or 48, wherein the redox active material is selected from the group consisting of poly luminol, methylene blue, ferrocene, ferrocene derivatives, polyluminol, 3,3′,5,5′-tetramethylbenzidine (TMB), o-phenylenediamine dihydrochloride (OPD), N,N,N′,N′-tetramethyl-p-phenylenediamine, viologens 2,2′-azino-bis(3-ethylbenzothiazoline-6-sulfonic acid) (ABTS), p-nitrophenyl Phosphate (PNPP), 3,3′-diaminobenzidine (DAB), 4-chloro-1-naphthol (4-CN), 5-bromo-4-chloro-3-indolyl-phosphate (BCIP), nitro blue tetrazolium (NBT), tetrathialfulvaene, 2,6-dichloroindophenol (DCIP), 2,6-dichloroindophenyl phosphate, riboflavin 5′-monophosphate (RMP), ethyl viologen (1,1′-bis(ethyl)-4,4′-bipyridinium or BEBP), Paraquat, hexaamine ruthenium, 1,4-naphthoquione, resorufine, cyanomethylviologen, diquat, triquat, 1,1′-bis(2-sulfoethyl)-4,4′-bipyridinium, 1,1′-dibenzyl-4,4′-bipyridinium, 4,4′-dicarboxy-2,2′-bipyridyl, 1-hydroxybenzotriazole, veratryl alcohol, violuric acid, 2-methoxy-phenothiazone,3-hydroxyanthranilic acid, anthraquinone 2,6-disulfonic acid, N-hydroxyacetanilide, phenol red, 3,3′,5,5′-tetramethyl benzidine, dichlorophenol red, 2,2′,6,6′-tetramethylpiperidine-N-oxyl radical, syringaldehyde and acetosyringone, 2,2,6,6-tetramethylpiperidin-1-yl)oxyl (TEMPO), 2,2′7,7′-tetrakis-(N,N-di-p-methoxyphenyl-amine)-9,9′-spirobifluorene (spiro-MeOTAD), sodium anthraquinone-2,6-di sulphonate (AQDS), benzoquinones, 2,2′-biimidazole, 2-(2-pyridyl)imidazole, 2,2′-bipyridine, tetracyanoquinodimethane (TCNQ), and combinations thereof.

Embodiment 50: The electrode of any one of Embodiments 47-49, wherein the redox active material is poly luminol, ferrocene, or methylene blue.

Embodiment 51: The electrode of any one of Embodiments 47-50, wherein the redox active material is embedded within the polymeric coating layer.

Embodiment 52: The electrode of any one of Embodiments 1-51, wherein the target-binding molecule is covalently linked to the surface of the conductive substrate.

Embodiment 53: The electrode of any one of Embodiments 1-52, wherein the target-binding molecule is linked to the surface of the conductive substrate via a linker.

Embodiment 54: A field-effect transistor (FET) comprising an electrode of any one of Embodiments 1-53.

Embodiment 55: A sensor comprising an electrode of any one of Embodiments 1-53 or a field-effect transistor of Embodiment 54.

Embodiment 56: The sensor of Embodiment 55, wherein the sensor comprises a fluid-contact surface and the electrode is immobilized on at least a portion of the fluid-contact surface.

Embodiment 57: The sensor of Embodiment 55 or 56, wherein the sensor comprises one or more microfluidic flow cells.

Embodiment 58: The sensor of any one of Embodiments 55-57, wherein the sensor comprises one or more microfluidic flow cells.

Embodiment 59: The sensor of any one of Embodiments 55-58, wherein the fluid-contact surface further comprises a positive control electrode and/or a negative control electrode immobilized thereon.

Embodiment 60: Use of an electrode of any one of Embodiments 1-53, or a field-effect transistor of Embodiment 54, or a sensor of any one of Embodiments 55-59 for detecting a target analyte in a sample.

Embodiment 61: A method for detecting a target analyte in a sample, the method comprising: contacting a sample suspected of comprising a target analyte with an electrode of any one of Embodiments 1-53 and detecting binding of the target analyte with the target binding ligand.

Embodiment 62: The method of Embodiment 61, wherein said detecting the binding of the target molecule with the target binding ligand comprises applying a voltage to the electrode.

Embodiment 63: The method of Embodiment 61 or 62, wherein said detecting the binding of the target molecule with the target binding ligand comprises measuring a current generated from electrode.

Embodiment 64: The method of any one of Embodiments 62 or 63, wherein said detecting the binding of the target molecule with the target binding molecule comprises contacting a second target binding molecule to the target molecule, wherein the second target binding molecule comprises a detectable label.

Embodiment 65: The method of Embodiment 64, wherein said contacting with the second target binding molecule is prior to contacting the sample with the electrode.

Embodiment 66: The method of Embodiment 64, wherein said contacting with the second target binding molecule is after contacting the sample with the electrode.

Embodiment 67: The method of any one of Embodiments 64-66, wherein the detectable label comprises an enzyme, a fluorophore, a chemiluminescent label, colloidal gold, colored glass or plastic (e.g., polystyrene, polypropylene, and latex) beads, a radiolabel, a quantum dot, or any combination thereof.

Embodiment 68: The method of any one of Embodiments 64-67, wherein the detectable label comprises an enzyme.

Embodiment 69: The method of Embodiment 68, wherein the enzyme is a peroxidase, alkaline phosphatase, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase or acetylcholinesterase.

Embodiment 70: The method of any one of Embodiments 68-69, wherein the method further comprises contacting the enzyme with a substrate of the enzyme.

Embodiment 71: The method of any one of Embodiments 64-70, wherein the detectable label facilitates generation of a charge carrier.

Embodiment 72: The method of Embodiment 71, wherein said detecting the binding of the target molecule with the target binding molecule comprises detecting the charge carrier.

T Embodiment 73: he method of any one of Embodiments 61-72, wherein the target analyte is a peptide, a polypeptide, a peptidomimetic, a nucleic acid, an oligosaccharide, a polysaccharide, an amino acid, nucleoside, a nucleotide, a carbohydrate, a lipid, a peptidoglycan, a cell, microbial matter, an antigen, a lipid, a steroid, a hormone, a lipopolysaccharide, an endotoxin, a therapeutic agent, a lipid-binding molecule, a cofactor, a small molecule, a toxin, a biological threat agent (e.g., spore, viral, cellular and protein toxin), or any combination thereof.

Embodiment 74: The method of any one of Embodiments 61-73, wherein the target analyte is a protein, an antibody, an antigen binding fragment of an antibody, an antigen, a hormone, or a metabolite.

Embodiment 75: The method of any one of Embodiments 61-74, wherein the target analyte is a tumor marker or a clinical chemistry target.

Embodiment 76: The method of any one of Embodiments 61-75, wherein the sample is a biological sample (e.g., blood, saliva, amniotic fluid, sputum, urine, semen, cerebrospinal fluid, bronchial aspirate, perspiration, mucus, liquefied stool sample, synovial fluid, lymphatic fluid, tears, tracheal aspirate, lactation product, breath condensate and any combination thereof).

Embodiment 77: The method of any one of Embodiments 61-76, wherein the sample is a food, an ingredient for preparing a food, poultry, meat, fish, beverage, grain (e.g., food grain), crop, or dairy product.

Embodiment 78: The method of any one of Embodiments 61-77, wherein the sample is a non-biological sample (e.g., water, salt water, pond water, river water, reservoir water, brine, drinking water, industrial water, brown water, waste water, sewerage, soil, and mixtures thereof.

Embodiment 79: The method of any one of Embodiments 61-78, wherein the sample is pre-processed prior to contacting with the electrode or the sensor.

Embodiment 80: A kit comprising an electrode of any one of Embodiments 1-53, or a field-effect transistor of Embodiment 54, or a sensor of any one of Embodiments 55-59.

Embodiment 81: A method for preparing an electrode comprising: (a) immobilizing a target binding molecule on a surface of a conductive substrate; and (b) forming or depositing a polymeric coating layer on said surface of the conductive substrate, and wherein the polymeric coating layer covers at least a non-target binding portion of the target-binding molecule.

Embodiment 82: The method of Embodiment 81, wherein a target-binding portion or site of the target-binding molecule is not covered by or embedded within the polymeric coating layer.

Embodiment 83: The method of Embodiment 81 or 82, wherein a target-binding portion or site of the target-binding molecule is exposed for contact with an analyte, and wherein said contact with the analyte is not within the polymeric coating layer.

Embodiment 84: The method of any one of Embodiments 81-83, wherein a target-binding portion or site of the target-binding molecule is at a surface of the polymeric coating layer.

Embodiment 85: The method of any one of Embodiments 81-84, wherein a target-binding portion or site of the target-binding molecule is at a surface of the polymeric coating layer, and wherein said surface of the polymeric coating layer is not a surface of a pore in the polymeric coating layer.

Embodiment 86: The method of any one of Embodiments 81-85, wherein the polymeric coating layer reduces or inhibits mobility ions in a liquid solution to the surface of the conductive substrate when the electrode is disposed in said liquid solution.

Embodiment 87: The method of any one of Embodiments 81-86, wherein the polymeric coating layer reduces or inhibits direct contact between ions in a liquid solution and the surface of the conductive substrate when the electrode is disposed in said liquid solution.

Embodiment 88: The method of any one of Embodiments 81-87, wherein the polymeric coating layer has a porosity of about 10% (e.g., about 9%, about 8%, about 7%, about 6%, about 5%, about 4%, about 3% about 2.5%, about 2%, about 1.5%, about 1%, about 0.75%, about 0.5%) or lower.

Embodiment 89: The method of any one of Embodiments 81-88, wherein the polymeric coating layer is substantially non-porous.

Embodiment 90: The method of any one of Embodiments 81-89, wherein the polymeric coating layer completely covers the surface of the conductive substrate.

Embodiment 91: The method of any one of Embodiments 81-90, wherein a thickness of the polymeric coating layer is about 95% or less of a distance between a target-binding portion or site of the target-binding molecule and the surface of the conductive substrate.

Embodiment 92: The method of any one of Embodiments 81-91, wherein a thickness of the polymeric coating layer is at least about 1 nm.

Embodiment 93: The method of any one of Embodiments 81-92, wherein a thickness of the polymeric coating layer is about 20 nm or lower.

Embodiment 94: The method of any one of Embodiments 81-93, wherein said forming or depositing the polymeric coating layer on the surface of the conductive substrate comprises electro-polymerization, photo-polymerization, or auto-polymerization, optionally, said forming or depositing the polymeric coating layer on the surface of the conductive substrate comprises electro-polymerization.

Embodiment 95: The method of any one of Embodiments 81-94, wherein the polymeric coating layer comprises a polymer selected from the group consisting of polysaccharides, polydopamines, poly-p-phenylenes, polypyrroles, coumarin based polymers, polyhydroxyethylmethacrylates, polyindolyenes, polyanilines, polythiophenes, poly(ethylenedioxy) thiophene, poly(heteroaromatic vinylenes), polyvinylphosphates, poly-p-phenylene sulfides, poly(2,5-thienylenes), poly-o-phenylenes, poly(o-phenylene diamines), polysaccharides, polyacetylenes, porphyrins, phthalocyanins, fullerenes, polyparaphenylenes, polyphenylenevinylenes, polyfluorenes, polypyridines, polycarbazoles, polypyridinevinylenes, polyarylvinylenes, poly (p-phenylmethylvinylenes), derivatives and co-polymers thereof, and any combination thereof.

Embodiment 96: The method of any one of Embodiments 81-95, wherein the polymeric coating layer comprises polydopamine, chitosan, or scopoletin (6-methoxy-7-hydroxycoumarin).

Embodiment 97: The method of any one of Embodiments 81-96, wherein said immobilizing the target binding molecule on the surface of the conductive substrate comprises covalently linking the target binding molecule on the surface of the conductive substrate.

Embodiment 98: The method of any one of Embodiments 81-97, wherein said immobilizing the target binding molecule on the surface of the conductive substrate comprises linking the target binding molecule on the surface of the conductive substrate via a linker.

Embodiment 99: The method of any one of Embodiments 81-98, wherein the target-binding molecule is a peptide, polypeptide, a nucleic acid (e.g., oligonucleotide or polynucleotide) or a small molecule.

Embodiment 100: The method of any one of Embodiments 81-99, wherein the target binding molecule is a receptor, a ligand for a receptor, an antibody, antigen binding fragment of an antibody, an antigen, an enzyme, an affimer or an aptamer.

Embodiment 101: The method of any one of Embodiments 81-100, wherein the target binding molecule is an antibody, an antigen binding fragment of an antibody, or an antigen, optionally, the target binding molecule is an antibody or an antigen binding fragment of an antibody.

Embodiment 102: The method of any one of Embodiments 81-101, further comprising adding an antifouling material to the polymeric coating layer.

Embodiment 103: The method of Embodiment 102, wherein said adding the antifouling material to the polymeric coating layer is prior to forming or depositing the polymeric layer on the surface of the conductive substrate.

Embodiment 104: The method of Embodiment 102 or 103, wherein the antifouling material is ethanolamine, hyaluronic acid, or poly vinyl alcohol.

Embodiment 105: The method of any one of Embodiments 81-104, wherein the electrode is a planar or 3-dimensional electrode.

Embodiment 106: The method of any one of Embodiments 81-105, wherein the conductive substrate comprises gold, silver, copper, platinum, aluminum, stainless steel, tungsten, indium tin oxide, titanium, lead, nickel, palladium, zirconium, niobium, tantalum, chromium, molybdenum, manganese, rhenium, ruthenium, rhodium, iridium, silicon, polyimide, parylene, benzocyclobutene, carbon, graphite, or any combination thereof.

Embodiment 107: The method of any one of Embodiments 81-106, wherein the conductive substrate comprises a flexible substrate.

Embodiment 108: The method of any one of Embodiments 81-107, wherein the conductive substrate comprises a flexible substrate selected from the group consisting of polyethylene terephthalate, polyethylene naphathalate, polyimides, polymeric hydrocarbons, celluloses, plastics, polycarbonates, polystyrenes, silicon and any combination thereof.

Embodiment 109: The method of any one of Embodiments 81-108, wherein the polymeric coating layer is adapted for contact with an analyte or a sample comprising an analyte.

Embodiment 110: The method of any one of Embodiments 81-109, wherein the electrode is an electrode of any one of Embodiments 1-53.

Additional exemplary aspects of the disclosure are described by one or more of following numbered Embodiments:

Embodiment 1: An electrode comprising: (i) a conductive substrate; (ii) a target-binding molecule covalently conjugated on a surface of the conductive substrate, wherein the target binding molecule is capable of binding with a target molecule; and (iii) and a polymeric coating layer comprising a polymer on said surface, and wherein at least a non-target binding portion of the target-binding is embedded within the polymeric coating layer.

Embodiment 2: The electrode of Embodiment 1, wherein the polymeric coating layer comprises an electropolymerized polymer, photopolymerized polymer, or auto-polymerized polymer.

Embodiment 3: The electrode of any one of Embodiment 1-2, wherein the polymer coating layer comprises a electropolymerized polymer.

Embodiment 4: The electrode of any one of Embodiment 1-3, wherein the polymeric coating layer comprises a polymer selected from the group consisting of polysaccharides, polydopamines, poly-p-phenylenes, polypyrroles, coumarin based polymers, polyhydroxyethylmethacrylates, polyindolyenes, polyanilines, polythiophenes, poly(ethylenedioxy) thiophene, poly(heteroaromatic vinylenes), polyvinylphosphates, poly-p-phenylene sulfides, poly(2,5-thienylenes), poly-o-phenylenes, poly(o-phenylene diamines), polysaccharides, polyacetylenes, porphyrins, phthalocyanins, fullerenes, polyparaphenylenes, polyphenylenevinylenes, polyfluorenes, polypyridines, polycarbazoles, polypyridinevinylenes, polyarylvinylenes, poly (p-phenylmethylvinylenes), derivatives and co-polymers thereof, and any combination thereof.

Embodiment 5: The electrode of any one of Embodiments 1-4, wherein the polymeric coating layer comprises polydopamine, chitosan, or scopoletin (6-methoxy-7-hydroxycoumarin).

Embodiment 6: The electrode of any one of Embodiment 1-5, wherein the polymeric coating layer comprises electropolymerized polydopamine, electropolymerized chitosan, electropolymerized scopoletin, or autopolymerized chitosan.

Embodiment 7: The electrode of any one of Embodiment 1-6, wherein the polymeric coating layer further comprises a dissolvable or degradable material.

Embodiment 8: The electrode of Embodiment 7, wherein the dissolvable or degradable material is selected from the group consisting of water dissolvable polymers (salt crystals), materials capable of being removed by degradation (e.g., proteins or protein aggregates), temperature dependent polymers (e.g., poly(N-isopropyl acrylamide) (PNIPAAm)); and any combination thereof.

Embodiment 9: The electrode of any one of Embodiments 1-8, wherein the polymeric coating layer further comprises an antifouling material.

Embodiment 10: The method of Embodiment 9, wherein the antifouling material is ethanolamine (ETA), hyaluronic acid (HA), or poly vinyl alcohol (PVA) Embodiment 11: The electrode of any one of Embodiments 1-10, wherein the target binding molecule is a receptor, a ligand for a receptor, an antibody, antigen binding fragment of an antibody, an antigen, an enzyme or a nucleic acid.

Embodiment 12 The electrode of one of Embodiments 1-11, wherein the target binding molecule is an antibody, an antigen binding fragment of an antibody or an antigen.

Embodiment 13: The electrode of any one of Embodiments 1-12, wherein the polymeric coating layer further comprises a conductive element.

Embodiment 14: The electrode of any one of Embodiments 1-13, wherein the electrode is a planar or 3-dimensional electrode.

Embodiment 15: The electrode of any one of Embodiments 1-14, wherein the conductive substrate comprises gold, silver, copper, platinum, aluminum, stainless steel, tungsten, indium tin oxide, titanium, lead, nickel, palladium, zirconium, niobium, tantalum, chromium, molybdenum, manganese, rhenium, ruthenium, rhodium, iridium, silicon, polyimide, parylene, benzocyclobutene, carbon, graphite, or any combination thereof.

Embodiment 16: The electrode of any one of Embodiments 1-15, wherein the conductive substrate comprises a flexible substrate.

Embodiment 17: The electrode of any one of Embodiments 1-16, wherein the conductive substrate comprises a flexible substrate selected from the group consisting of polyethylene terephthalate, polyethylene naphathalate, polyimides, polymeric hydrocarbons, celluloses, plastics, polycarbonates, polystyrenes, silicon and any combination thereof.

Embodiment 18 The electrode of any one of Embodiments 1-17, wherein the polymeric coating layer is adapted for contact with an analyte or a sample comprising an analyte.

Embodiment 19: The electrode of any one of Embodiments 1-18, wherein the polymeric coating layer is porous.

Embodiment 20: The electrode of any one of Embodiments 1-19, wherein the polymeric coating layer has a porosity of about 5% to about 95%.

Embodiment 21: The electrode of any one of Embodiments 1-20, wherein the polymeric coating layer comprises macropores (e.g., pores having a diameter of from about 0.1 um to about 10 μm such as from about 0.5 μm to about 5 μm or from about 1 μm to about 3 μm).

Embodiment 22: The electrode of any one of Embodiments 1-21, wherein the polymeric coating layer comprises mesopores (e.g., pores having a diameter from about 5 nm to about 99 nm).

Embodiment 23: The electrode of any one of Embodiments 1-22, wherein the polymeric coating layer comprises nanopores (e.g., pores having a diameter of from about 0.1 nm to about 4.5 nm).

Embodiment 24: The electrode of any one of Embodiments 1-23, wherein the polymeric coating layer further comprises a conducting material.

Embodiment 25: The electrode of Embodiment 24, wherein the conducting material comprises conductive particles, conductive rods, conductive fibers, conductive nano-particles, conductive polymers, conductive nano-flakes, conductive nanotubes, semi-conductive particles, semi-conductive rods, semi-conductive fibers, semi-conductive nano-particles, semi-conductive nanoflakes, semi-conductive nanotubes, or semi-conductive polymers.

Embodiment 26: The electrode of Embodiment 24 or 25, wherein the conducting material is a metal, a metalloid, conducting polymer, a conducting carbon-based material, organic compounds having conducting and/or semiconducting properties, or any combination thereof.

Embodiment 27: The electrode of any one of Embodiments 24-26, wherein the conducting material comprises an allotrope of carbon atoms arranged in a hexagonal lattice.

Embodiment 28: The electrode of any one of Embodiments 24-27, wherein the conducting material is graphite, functionalised graphene oxide, reduced graphene oxide, or carbon nano-tubes (CNTs).

Embodiment 29 The electrode of Embodiment 28, wherein the carbon nanotubes are carboxylated carbon nanotubes or aminated carbon nanotubes.

Embodiment 30: The electrode of Embodiment 28, wherein the reduced graphene oxide is a carboxylated reduced graphene oxide or an aminated reduced graphene oxide.

Embodiment 31: The electrode of any one of Embodiments 24-26, wherein the conductive material comprises gold.

Embodiment 32: The electrode of any one of Embodiments 1-31, wherein the polymeric coating layer further comprises a redox active material.

Embodiment 33: The electrode of Embodiment 32, wherein the redox active material is selected from the group consisting of metallocenes metallocence derivatives, quinones, polycyclic aromatic hydrocarbons, redox-active organic molecules, phenazines, organosulfur compounds, and radical acceptors.

Embodiment 34: The electrode of any one of Embodiments 32-33, wherein the redox active material is selected from the group consisting of poly luminol, methylene blue, ferrocene, ferrocene derivatives, polyluminol, 3,3′,5,5′-tetramethylbenzidine (TMB), o-phenylenediamine dihydrochloride (OPD), N,N,N′,N′-tetramethyl-p-phenylenediamine, viologens 2,2′-azino-bis(3-ethylbenzothiazoline-6-sulfonic acid) (ABTS), p-nitrophenyl Phosphate (PNPP), 3,3′-diaminobenzidine (DAB), 4-chloro-1-naphthol (4-CN), 5-bromo-4-chloro-3-indolyl-phosphate (BCIP), nitro blue tetrazolium (NBT), tetrathialfulvaene, 2,6-dichloroindophenol (DCIP), 2,6-dichloroindophenyl phosphate, riboflavin 5′-monophosphate (RMP), ethyl viologen (1,1′-bis(ethyl)-4,4′-bipyridinium or BEBP), Paraquat, hexaamine ruthenium, 1,4-naphthoquione, resorufine, cyanomethylviologen, diquat, triquat, 1,1′-bis(2-sulfoethyl)-4,4′-bipyridinium, 1,1′-dibenzyl-4,4′-bipyridinium, 4,4′-dicarboxy-2,2′-bipyridyl, 1-hydroxybenzotriazole, veratryl alcohol, violuric acid, 2-methoxy-phenothiazone, 3-hydroxyanthranilic acid, anthraquinone 2,6-disulfonic acid, N-hydroxyacetanilide, phenol red, 3,3′,5,5′-tetramethyl benzidine, dichlorophenol red, 2,2′,6,6′-tetramethylpiperidine-N-oxyl radical, syringaldehyde and acetosyringone, 2,2,6,6-tetramethylpiperidin-1-yl)oxyl (TEMPO), 2,2′7,7′-tetrakis-(N,N-di-p-methoxyphenyl-amine)-9,9′-spirobifluorene (spiro-MeOTAD), sodium anthraquinone-2,6-di sulphonate (AQDS), benzoquinones, 2,2′-biimidazole, 2-(2-pyridyl)imidazole, 2,2′-bipyridine, tetracyanoquinodimethane (TCNQ), and combinations thereof.

Embodiment 35: The electrode of any one of Embodiments 32-34, wherein the redox active material is poly luminol, ferrocene, or methylene blue.

Embodiment 36: The electrode of any one of Embodiments 1-35, wherein the redox active material is embedded within the antifouling coating layer.

Embodiment 37: A sensor comprising an electrode of any one Embodiments 1-36.

Embodiment 38: The sensor of Embodiment 37, wherein the sensor comprises a fluid-contact surface and the electrode is immobilized on at least a portion of the fluid-contact surface.

Embodiment 39: The sensor of Embodiment 37 or 38, wherein the sensor comprises one or more microfluidic flow cells.

Embodiment 40: The sensor of any one of Embodiments 37-39, wherein the sensor comprises one or more microfluidic flow cells.

Embodiment 41: The sensor of any one of Embodiments 37-40, wherein the fluid-contact surface further comprises a positive control electrode and/or a negative control electrode immobilized thereon.

Embodiment 42: Use of an electrode of any one of Embodiments 1-36 or sensor of any one of Embodiments 37-41 or 63 for detecting a target analyte in a sample.

Embodiment 43: A method for detecting a target analyte in a sample, the method comprising: contacting a sample suspected of comprising a target analyte with an electrode of any one of Embodiments 1-36 and detecting binding of the target analyte with the target binding ligand.

Embodiment 44: The method of Embodiment 43, wherein said detecting the binding of the target molecule with the target binding ligand comprises applying a voltage to the electrode.

Embodiment 45: The method of Embodiment 43 or 43, wherein said detecting the binding of the target molecule with the target binding ligand comprises measuring a current generated from electrode.

Embodiment 46: The method of any one of Embodiments 44-45, wherein said detecting the binding of the target molecule with the target binding molecule comprises contacting a second target binding molecule to the target molecule, wherein the second target binding molecule comprises a detectable label.

Embodiment 47: The method of Embodiment 46, wherein said contacting with the second target binding molecule is prior to contacting the sample with the electrode.

Embodiment 48: The method of Embodiment 46, wherein said contacting with the second target binding molecule is after contacting the sample with the electrode.

Embodiment 49: The method of any one of Embodiments 46-47, wherein the detectable label comprises an enzyme, a fluorophore, a chemiluminescent label, colloidal gold, colored glass or plastic (e.g., polystyrene, polypropylene, and latex) beads, a radiolabel, a quantum dot, or any combination thereof.

Embodiment 50: The method of any one of Embodiments 46-49, wherein the detectable label comprises an enzyme.

Embodiment 51: The method of Embodiment 50, wherein the enzyme is a peroxidase, alkaline phosphatase, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase or acetylcholinesterase.

Embodiment 52: The method of any one of Embodiments 49-51, wherein the method further comprises contacting the enzyme with a substrate of the enzyme.

Embodiment 53: The method of any one of Embodiments 47-50, wherein the detectable label facilitates generation of a charge carrier.

Embodiment 54: The method of Embodiment 53, wherein said detecting the binding of the target molecule with the target binding molecule comprises detecting the charge carrier.

Embodiment 55: The method of any one of Embodiments 43-54, wherein the target analyte is a peptide, a polypeptide, a peptidomimetic, a nucleic acid, an oligosaccharide, a polysaccharide, an amino acid, nucleoside, a nucleotide, a carbohydrate, a lipid, a peptidoglycan, a cell, microbial matter, an antigen, a lipid, a steroid, a hormone, a lipopolysaccharide, an endotoxin, a therapeutic agent, a lipid-binding molecule, a cofactor, a small molecule, a toxin, a biological threat agent (e.g., spore, viral, cellular and protein toxin), or any combination thereof.

Embodiment 56: The method of any one of Embodiments 43-55, wherein the target analyte is a protein, an antibody, an antigen binding fragment of an antibody, an antigen, a hormone, or a metabolite.

Embodiment 57: The method of any one of Embodiments 43-56, wherein the target analyte is a tumor marker or a clinical chemistry target.

Embodiment 58: The method of any one of Embodiments 43-57, wherein the sample is a biological sample (e.g., blood, saliva, amniotic fluid, sputum, urine, semen, cerebrospinal fluid, bronchial aspirate, perspiration, mucus, liquefied stool sample, synovial fluid, lymphatic fluid, tears, tracheal aspirate, lactation product, breath condensate, and any combination thereof).

Embodiment 59: The method of any one of Embodiments 43-58, wherein the sample is a food, an ingredient for preparing a food, poultry, meat, fish, beverage, grain (e.g., food grain) crop, or dairy product.

Embodiment 60: The method of any one of Embodiments 43-59, wherein the sample is a non-biological sample (e.g., water, salt water, pond water, river water, reservoir water, brine, drinking water, industrial water, brown water, waste water, sewerage, soil, and mixtures thereof.

Embodiment 61: The method of any one of Embodiments 43-60, wherein the sample is pre-processed prior to contacting with the electrode or the sensor.

Embodiment 62: A kit comprising an electrode of any one of Embodiments 1-36, sensor of any one of Embodiments 37-41 or 64, or field-effect transistor (FET) of Embodiment 63.

Embodiment 63: A field-effect transistor (FET) comprising an electrode of any one of Embodiments 1-36.

Embodiment 64: A sensor comprising the field effect transistor of Embodiment 63.

### Some Selected Definitions

For convenience, certain terms employed herein, in the specification, examples and appended claims are collected herein. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

As used herein the term “comprising” or “comprises” is used in reference to compositions, methods, and respective component(s) thereof, that are essential to the invention, yet open to the inclusion of unspecified elements, whether essential or not. In other words, except where the context requires otherwise, the term “comprise” and variations of the term, such as “comprising”, “comprises” and “comprised”, are not intended to exclude further additives, components, integers or steps.

The singular terms “a,” “an,” and “the” include plural referents unless context clearly indicates otherwise. Similarly, the word “or” is intended to include “and” unless the context clearly indicates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

As used herein, the term “binding” or “bound” generally refers to a reversible binding of one molecule to molecule via, e.g., van der Waals force, hydrophobic force, hydrogen bonding, and/or electrostatic force. The binding interaction between two molecules can be described by a dissociation constant (K_{d}) or association constant (K).

Specific elements of any of the disclosed embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

### EXAMPLES

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### Example 1: Label-Free Electrochemical Sensing Enabled by a Thin Polymeric Layer to Circumvent Debye Length Screening

Point-of-care sensors demonstrate significant promise for at-home and patient-centric diagnostics. However, most of them are label-based that involve multiple laborious steps, reagents, limited shelf-life and require to be performed by trained personnel and have reduced stability. One potential approach is to develop label-free sensing platforms which utilize a single molecular binding event (e.g., only one antibody-antigen binding event). Such an approach reduces the number of steps, enables direct readout of binding events, uses fewer reagents, and simplifies the product development steps.

However, such an approach has been limited to R&D settings like surface plasmon resonance techniques (SPR). For electrochemical sensing, label-free sensing is limited because of the screening of charges beyond the Debye length, which decreases the sensitivity of the sensors. The innovation of the proposed technology is the development of a sensor stack with a polymer coating that offers higher sensitivity, stability and enhanced response for a wide range of biomarkers, including proteins and metabolites. The fabrication procedure of the sensor technology is a simple two-step process that will enable bulk manufacturing capability. Furthermore, as this technology measures the biomarker levels directly, this reduces assay steps and the associated cost significantly. Primarily, this sensing system is scalable due to the simplistic sensor fabrication and can be translated for at-home monitoring as it does not require trained personnel for sample measurement.

Conventional label-based sensing systems involve several steps and require trained personnel to detect target biomarkers. Electrochemical label-free assays demonstrate a plethora of potential for rapid and point-of-care diagnostics because of their ease of use, the ability for bulk manufacturing and portable form factor at a lost cost. However, one of the key challenges that has impeded the growth of electrochemical label-free sensors is the need to overcome charge-screening effects at the electrode/solution interface that otherwise can result in loss in sensitivity of the signal.

Typically, the binding interactions between the capture probe and target analyte result in a charge modulation of the electrical double-layer interface captured using electrochemical transduction. However, charge screening effects due to the reduced Debye length at the electrode/solution interface cause the binding interactions to occur outside the electrical double layer (EDL), thereby reducing the signal response. Enhancing the Debye length will aid in enhanced charge capacity at the electrode/solution interface, allowing binding interactions between the capture probe and target analyte to occur within the EDL. As a result, the measured electrochemical response is directly proportional to the target analyte concentration.

The inventors here report a novel approach to enhance the electrochemical signal of a label-free assay. The designed system involves a simple, single-step modification of the sensor surface through electropolymerization to achieve an enhanced signal response without requiring additional sample processing steps.

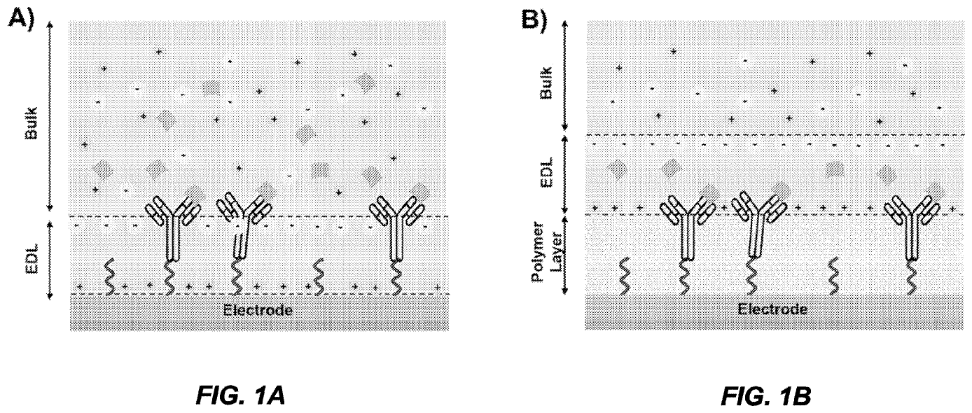

A schematic of the developed electropolymerized sensing system in comparison to no polymerization is demonstrated in FIGS. 1A and 1B. The electrode/solution interface without modification has a reduced EDL resulting in charge-screening, which causes a loss in signal response (FIG. 1A). However, the modification of the sensor interface with Dopamine through electropolymerization has an enhanced EDL preventing any charge-screening (FIG. 1B).

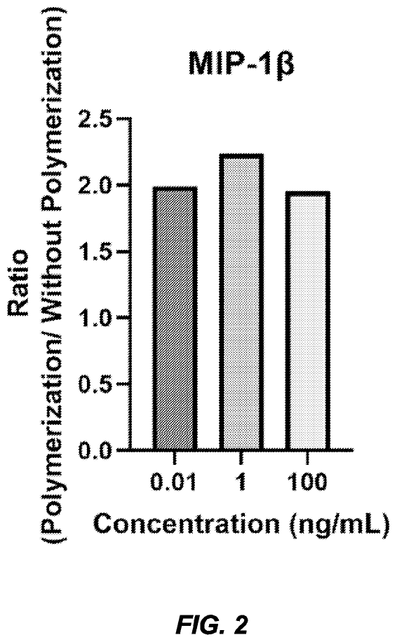

Preliminary experiments were performed to validate the label-free polymer-based sensor integrated with electrochemical readouts. The fabrication of the sensor has been described in detail in Zupancic, et al. Gold electrodes were fabricated on a glass wafer using standard photolithography process by depositing 20 nm of titanium and 150 nm of gold on a glass wafer, as described previously. The gold chips were then cleaned in acetone, followed by isopropanol for 5 min through sonication. The chips were then treated with oxygen plasma to ensure a clean surface at 0.5 mbar and 50% power for 2 min prior to use. A thiol cross-linker, DSP (dithiobis (succinimidyl propionate)) (Thermo Fisher Scientific, USA, 22585) of 10 mM concentration was added to the sensor surface and incubated for 30 minutes for enabling covalent linkage of the antibody. MIP antibody (R&D systems, USA) of 1 mg/mL was immobilized for 2 hours. Post-immobilization of antibody, dopamine (Sigma Aldrich, USA) was electropolymerized using cyclic voltammetry (3 cycles, −0.5−0.5 V, 20 mV/s) on the sensor surface to enhance the electrical double layer. The electropolymerized sensors were then compared against sensors without electropolymerization (control) for various concentrations of MIP. The electropolymerized sensors show 2 times higher response than the control samples (FIG. 2).

### REFERENCES

1. Zupanc̆ic̆, U., Jolly, P., Estrela, P., Moschou, D., & Ingber, D. E. (2021). Graphene Enabled Low-Noise Surface Chemistry for Multiplexed Sepsis Biomarker Detection in Whole Blood. Advanced Functional Materials, 31(16), 2010638.

### Example 2: A Polymer-Based Diagnostic Sensing System for Label-Free Detection of Biomarkers

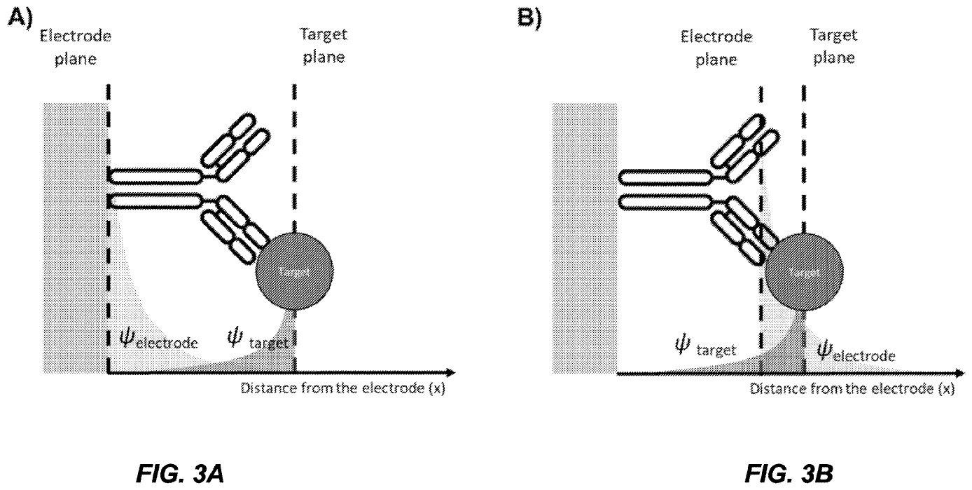

This study demonstrates that it is possible, by exploiting fundamental physics, to design electrode/electrolyte interfaces that enhance Debye length mitigating charge screening as well as allowing all biomolecular binding interactions between the capture probe and target analyte to occur within the EDL as shown in FIGS. 3A and 3B.

This work describes a novel surface-engineered approach with integrated polymer chemistry and biological probes to enhance the electrochemical signal response from direct biomolecular binding events in a label-free biosensor. This particular sensor design construct is achieved through a simple, single-step surface modification that encompasses biological probes via electropolymerization. The novel integration enables direct detection of target analytes in real time with exquisite sensing characteristics by overcoming charge screening that opens up new horizons for commercial realization of label-free electrochemical sensors. A schematic representation of the developed electropolymerized sensing system in comparison to no polymerization is shown schematically in FIGS. 1A and 1B. As shown, an unmodified electrode surface has reduced EDL resulting in charge screening that attenuates signal response (FIG. 3A) and polymer modified electrode surface showing reduced charge screening and enhanced EDL response (FIG. 3B).

### Sensor Fabrication

Preliminary experiments were performed to validate the label-free polymer-based sensor integrated with electrochemical readouts. The fabrication of the sensor has been described in detail in Zupanc̆ic̆, et al. Gold electrodes were fabricated on a glass wafer using standard photolithography process by depositing 20 nm of titanium and 150 nm of gold on a glass wafer, as described previously. The gold chips were then cleaned in acetone followed by isopropanol for 5 min through sonication. The chips were then treated with oxygen plasma to ensure a clean surface at 0.5 mbar and 50% power for 2 min prior to use. A thiol cross-linker, DSP (dithiobis (succinimidyl propionate)) (Thermo Fisher Scientific, USA, 22585) of 10 mM concentration was added to the sensor surface and incubated for 30 minutes for enabling covalent linkage of the antibody. MIP antibody (R&D systems, USA) of 1 mg/ml was immobilized for 2 hours. Post antibody immobilization, electropolymerization was performed using cyclic voltammetry to enhance EDL.

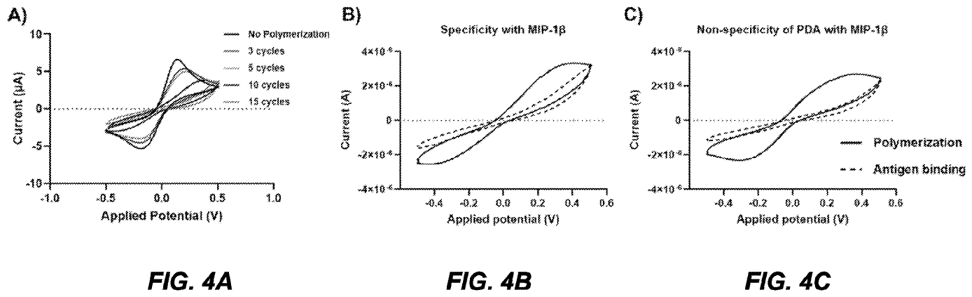

Dopamine: Dopamine undergoes polymerization at alkaline pH to form polydopamine, which can be formed on different electrode surfaces. The formation of polydopamine can be triggered by autoxidation that may result in more heterogenous and less organized films, which could negatively impact the sensor sensitivity. Thus, to ensure electrochemical-driven polymerization, the prepared dopamine solution was deaerated by passing nitrogen for 10 minutes. Polydopamine films were formed on antibody immobilized electrode surfaces by performing cyclic voltammetry (CV) from −0.5 to +0.5 V at the scan rate of 10 mV/s for 5 cycles. The thickness of polymer layer can be controlled by altering the number of CV cycles. As CV cycling proceeds, more dopamine gets deposited at the electrode surface resulting in the formation of insulative polydopamine coating at the working electrode surface that decreases current response (FIGS. 4A-4C). Specificity and non-specificity of polydopamine film was established with using MIP-1β as target analyte. Due to its adhesive properties, the active-NH₂ sites in the polymer attract interferents resulting in increased non-specificity of about ~55% in comparison to specific signal response (FIGS. 4A-4C).

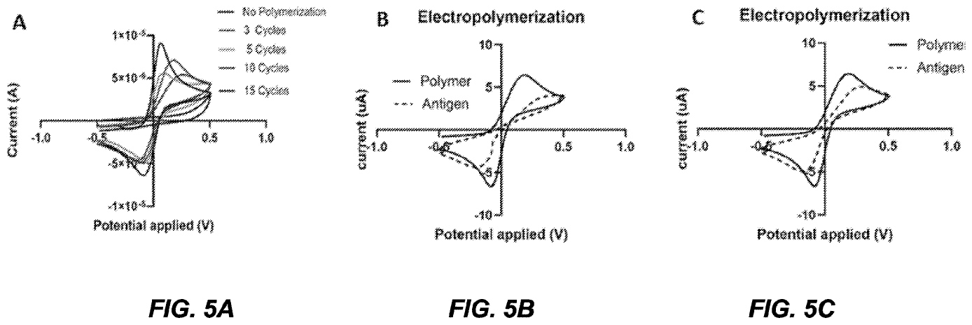

Chitosan: Chitosan is a naturally occurring polymer with linear arrangements of randomly distributed β-linked D-glucosamine and N-acetyl-D-glucosamine. The Chitosan is soluble in 0.1 M acetic acid and can electropolymerized at pH 5. The applied current leads to the electrodeposition by accumulating the charged chitosan macromolecules eventually forming a film like structure at the electrode surface (FIGS. 5A-5C). The chitosan was electropolymerized by employing CV with a voltage ranging from −0.2 V to +0.5 V at a constant scan rate of 80 mV. The electrodeposition thickness was controlled by varying the number of CV cycles as 3, 5, 10 and 15 forming a polymeric layer predominantly with hydrogen bonding and electrostatic interactions. The number of cycles correspond with deposition of chitosan and forming a insulative layer corroborating the flattening of the current response with each cycle. The specificity and non-specificity of the polymer was evaluated using IL-6 cytokine as a target analyte. The electrodeposited chitosan has a net charge that results in ~30% non-specific signal as compared to the specific signal response.

Scopoletin: Scopoletin, characterized by its electrical insulating properties and the absence of amine groups. This feature renders scopoletin less prone to non-specific binding. Similar to the previously examined polymers, the formation of polyscopoletin on the working electrode can be selectively achieved through cyclic voltammetry (CV). Critical to achieving a uniform layer that avoids covering the capture Ab epitope, minimizes charge screening, and enhances sensitivity, is the optimization of cycle number and scan rate.

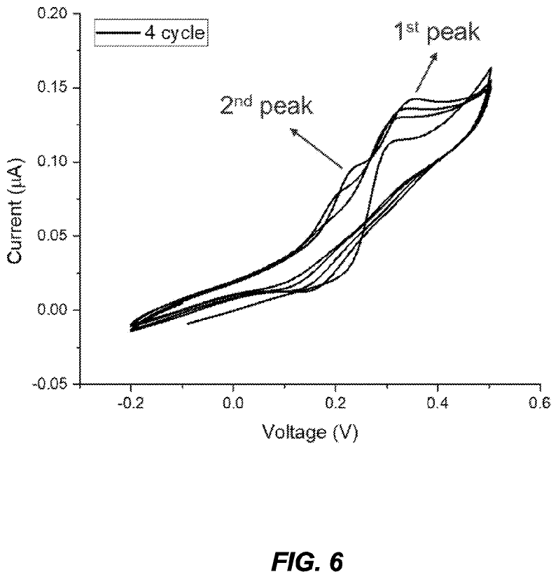

In cyclic voltammetry, the number of cycles (1~30) and the scan rate (10 and 500 m V/s.) were tested. These polymerization conditions varied depending on the buffer used for preparing scopoletin solution. We used the buffer of 0.1 M NaCl and 1% DMSO in DI water, both demonstrating a stable polymerization process at 0.1 mM scopoletin. During the CV-based polymerization, two distinct redox peaks were observed as the cycles progressed. The first involves the exchange of a single electron by scopoletin molecules, followed by a second redox reaction with an intermediate product (FIG. 6).

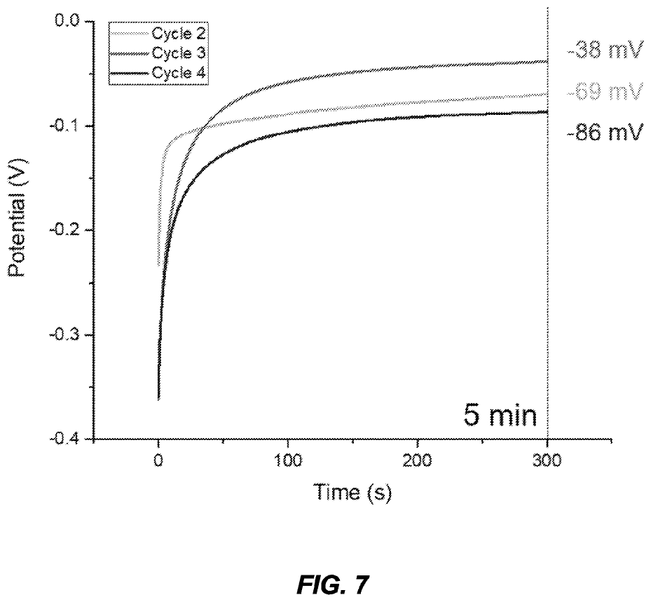

A pivotal consideration lies in the thermodynamic stability of polyscopoletin within the buffer. Without ensuring thermodynamic stability, there exists a risk of detachment of polymer layer from electrode over time, potentially leading to long-term stability issues. To address this concern, an electrochemical tool, the open circuit potential (OCP), was introduced (FIG. 7). The OCP serves as a means to assess the thermodynamic stability of the layer formed on and around the electrode. If the OCP saturates within the millivolt range within 30 minutes, it indicates that the electrode is thermodynamically stable; otherwise, it is deemed unstable. Upon measuring the OCP of polyscopoletin, convergence in tens of millivolts was observed in all cycles within 5 minutes, signifying excellent thermodynamic stability. This outcome is crucial for ensuring long-term stability.

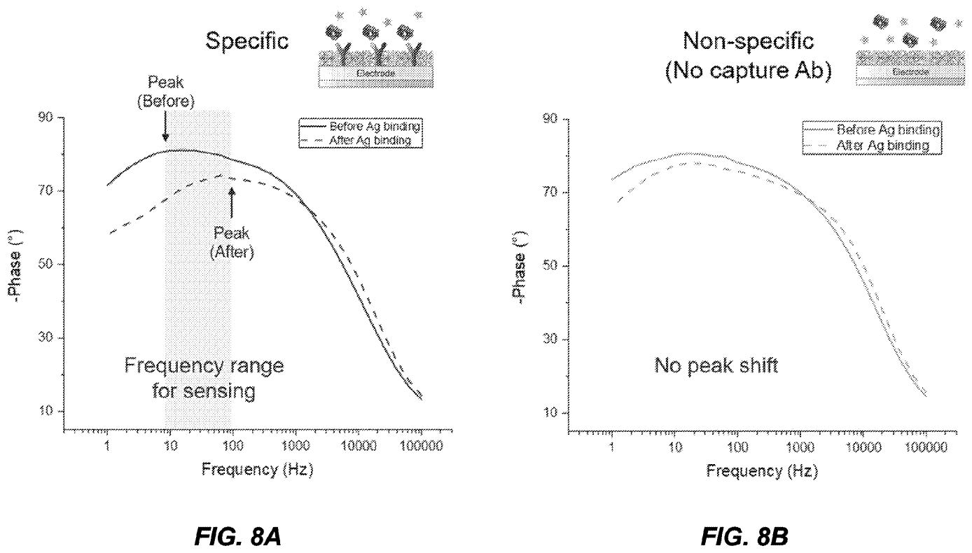

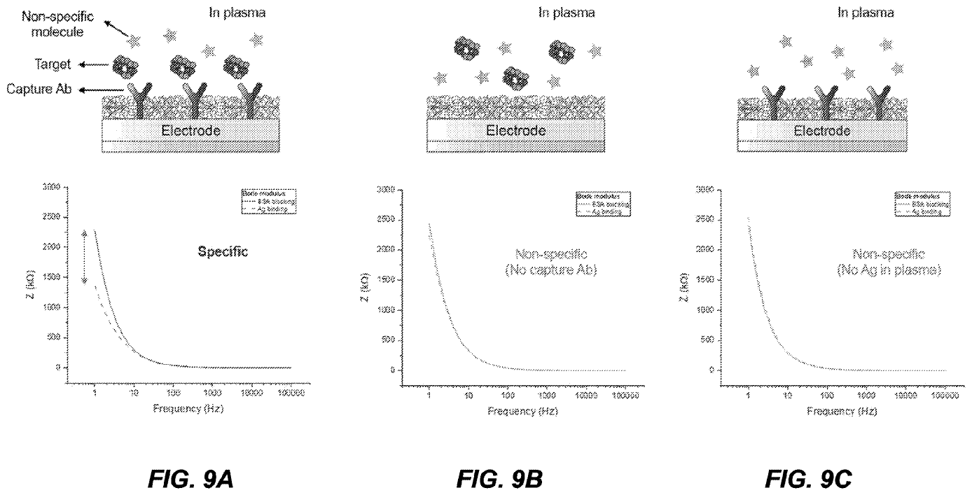

To assess the efficacy of the stable polyscopoletin for detecting target analytes in complex samples, MIP was spiked into plasma, and their potential for label-free sensing was explored. Electrochemical Impedance Spectroscopy (EIS) is an appropriate technique for label-free sensing in the absence of a redox marker, measuring the impedance change during the antibody-antigen binding. Within EIS analysis, two primary plots are considered: the phase plot and the Bode plot. Using the phase plot, we can identify a specific frequency range corresponding to antibody-antigen binding. Employing this frequency, we can quantify the change in impedance as a signal response in the Bode plot. FIGS. 8A and 8B illustrate the phase plot for specific and non-specific MIP-1β assay. MIP-1β was spiked in human plasma. Number of cycles (8 cycles) and scan rate (100 mv/s) were optimized. 0.1 mM scopoletin in 0.1M NaCl was used for this assay. Notably, a peak shift is observed in the specific sample, indicating the specific binding of antibody and antigen (FIG. 8A). Conversely, there is no peak shift in the non-specific samples (FIG. 8B). Utilizing the specific frequency range identified from the phase plot, we proceed to analyze impedance changes through the bode plot (FIGS. 9A-9C). To address the non-specificity issue, a comparative analysis was conducted under one specific and two non-specific conditions (No capture Ab and No target MIP in plasma). All measurements were performed in PBS without a redox marker. The graph illustrates a substantial disparity in the impedance change along the y-axis between the specific and non-specific samples. This distinction can be quantified at a specific frequency. Through these results, our technology demonstrates the capability to directly and sensitively detect the target analyte in complex biological fluids such as plasma, without the presence of any redox marker.

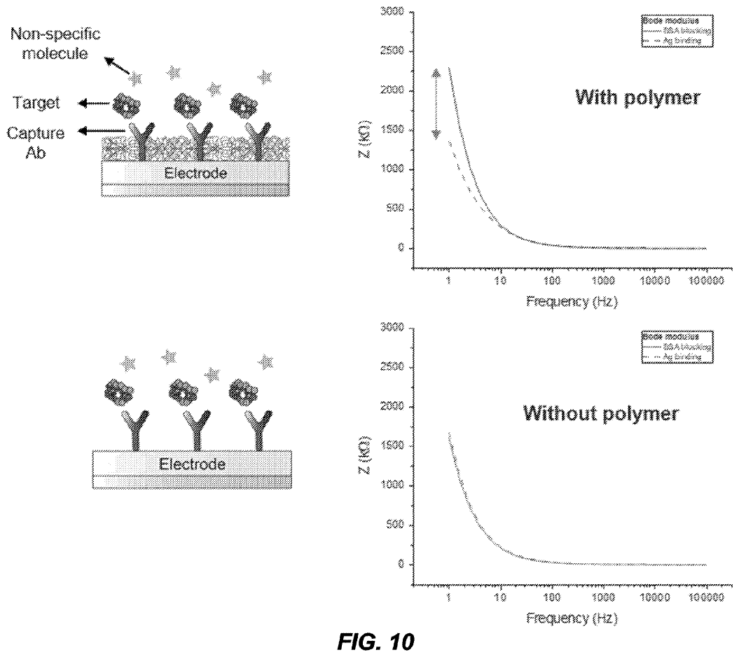

Changes in impedance based on the presence of scopoletin were observed to ascertain how much the our technology can amplify signals in label-free sensing. An MIP assay was conducted with and without the polymer (FIG. 10). As depicted in the Bode plot, there is minimal change in impedance without the polymer; however, in the sensor incorporating polyscopoletin, a substantial change in impedance is evident. This demonstrates that reducing charge screening has a considerable impact on sensitivity enhancement.

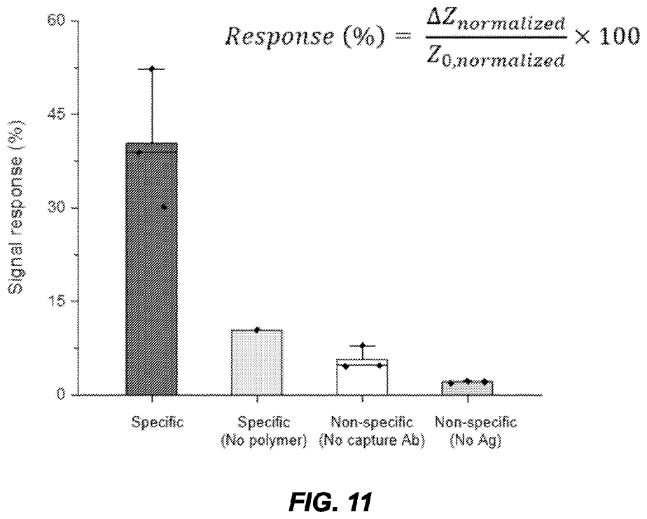

Using the aforementioned data, the change in impedance at a frequency of 10 Hz was calculated (FIG. 11). The specific sample exhibited a sensitivity approximately 7 times higher than the non-specific sample. Additionally, it showcased a sensitivity improvement of approximately 4 times compared to the sample without the polymer.

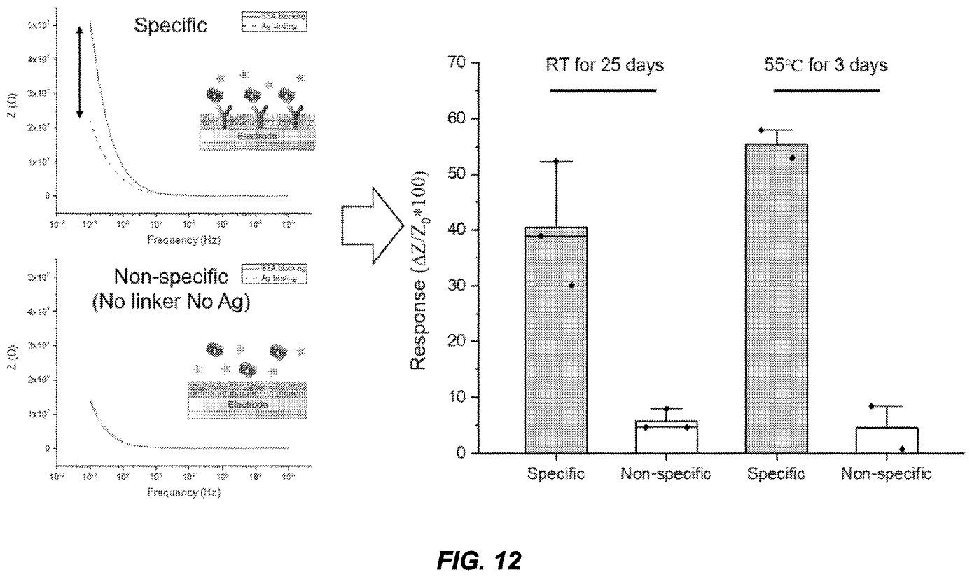

Scopoletin exhibits high solubility in DMSO, while its solubility is relatively low in the 0.1M NaCl solution used in this study. This solubility directly affects polymerization, thereby influencing sensor performance. To address this issue, a solution aging test was carried out (FIG. 12). An accelerated aging calculator was employed, and the solution was stored at 55° C. for three days. The resulting Bode plots depict specific and non-specific samples. As evident in the graphs, a significant and meaningful specific signal in impedance was achieved, surpassing the signal response observed in the solution stored at room temperature. Without wishing to be bound by a theory, the aging process of the solution contributes to a more stable formation of polyscopoletin on the electrode, thereby enhancing the performance of label-free sensing.

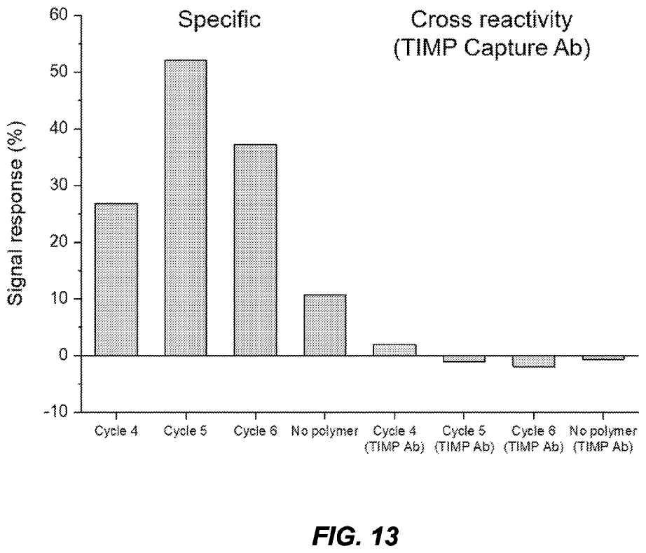

Furthermore, scopoletin solution was prepared in 1% DMSO in DI water, which shows higher solubility, and conducted MIP assay using this solution. As a result, in the 1% DMSO in DI water, the highest specific signal was observed when scopoletin is polymerized using CV of 5 cycles (FIG. 13). Additionally, when TIMP Ab was immobilized on the electrode and cross-reactivity was analyzed against MIP Ag, it exhibited a much lower signal compared to the specific signal. This confirms that scopoletin can be reliably formed on the electrode through various buffers, further securing a high specific signal for the target molecule.

### Polymer Composites

Generally, the active polymer sites are used for protein binding on to its surfaces. Therefore, it is necessary to block these active sites, which if not may contribute to non-specific interactions with other biomolecules in the biological samples.

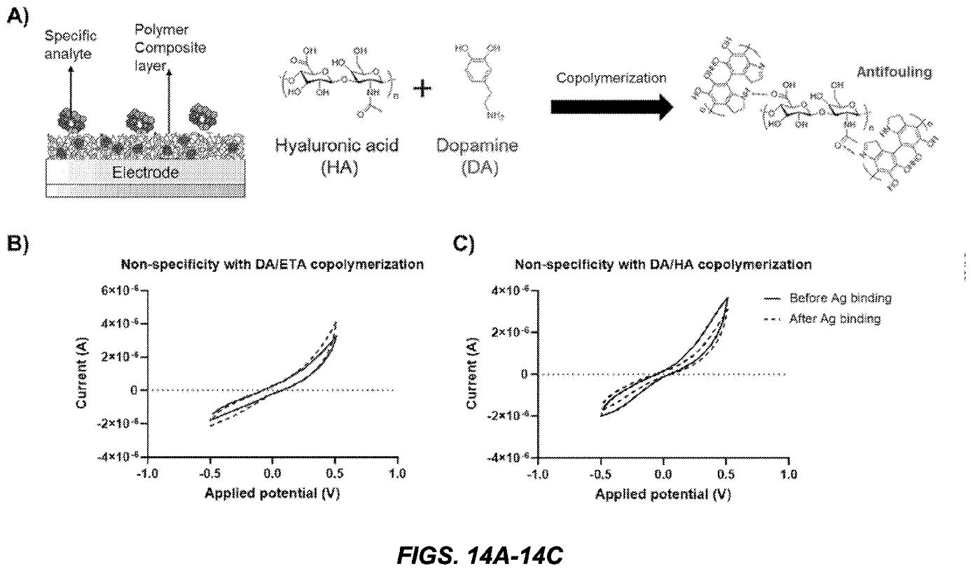

Polydopamine composites: To block the active —NH₂ sites of polydopamine, different antifouling materials, like ethanolamine (ETA) and hyaluronic acid (HA), were investigated. The incorporation of either ETA or HA in to polydopamine matrix gives a negatively charged surface resulting from the enrichment of surface with hydroxyl (—OH) functional groups, known to prevent non-specific adsorption (FIG. 14A). To evaluate antifouling capability of copolymerized polydopamine/ETA and polydopamine/HA surfaces, 10 ng/ml of MIP-1β was incubated on polymerized surfaces for an hour and CV measurements were performed before and after target analyte incubation. The results show reduction in non-specificity by about ~85%, which could be improved with further optimization of the polymer composition (FIGS. 14B and 14° C.).

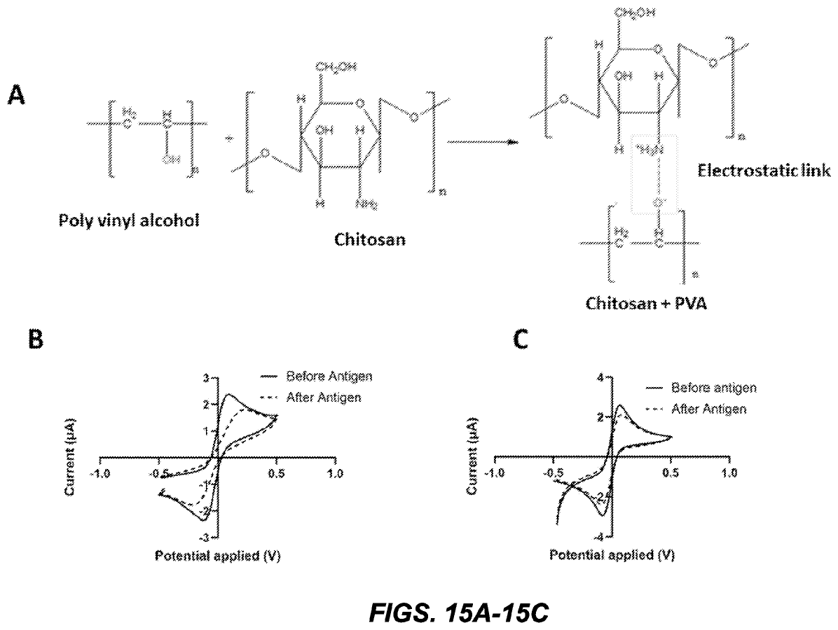

Chitosan composites: To block the active —NH2 sites of chitosan, neutralizing molecules such poly vinyl alcohol (PVA) was investigated. The —OH groups of PVA form an electrostatic link with chitosan masking the positively charged —NH₂ groups giving a relatively no net charge surface (FIG. 15A). To evaluate antifouling capability of copolymerized chitosan+PVA, 50 ng/ml of IL-6 in plasma was incubated on polymerized surfaces for an hour and CV measurements were performed before and after target analyte incubation. The results show about 80% reduction in non-specificity, which could be improved with further optimization of the polymer composition (FIGS. 15B and 15C).

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. An electrode comprising: (i) a conductive substrate; (ii) a target-binding molecule immobilized on surface of the conductive substrate, wherein the target-binding molecule is capable of binding with a target molecule; and (iii) and a polymeric coating layer comprising a polymer on said surface of the conductive substrate, and wherein the polymeric coating layer covers at least a non-target binding portion of the target-binding molecule.

2. The electrode of claim 1, wherein an electric double layer (EDL) is closer to a target-binding portion or site of the target-binding molecule than the surface of the conductive substrate.

3. The electrode of claim 1 or 2, wherein the polymeric coating layer forms an insulating or non-conducting layer between a target-binding portion or site of the target-binding molecule and the surface of the conductive substrate.

4. The electrode of any one of claims 1-3, wherein a target-binding portion or site of the target-binding molecule is not embedded within the polymeric coating layer.

5. The electrode of any one of claims 1-3, wherein a target-binding portion or site of the target-binding molecule is exposed for contact with an analyte, and wherein said contact with the analyte is not within the polymeric coating layer.

6. The electrode of any one of claims 1-3, wherein a target-binding portion or site of the target-binding molecule is at a surface of the polymeric coating layer.

7. The electrode of any one of claims 1-3, wherein the polymeric coating layer does not cover a target-binding portion or site of the target-binding molecule.

8. The electrode of any one of claims 1-7, wherein a target-binding portion or site of the target-binding molecule is at a surface of the polymeric coating layer, and wherein said surface of the polymeric coating layer is not a surface of a pore in the polymeric coating layer.

9. The electrode of any one of claims 1-8, wherein the polymeric coating layer reduces or inhibits mobility ions in a liquid solution to the surface of the conductive substrate when the electrode is disposed in said liquid solution.

10. The electrode of any one of claims 1-9, wherein the polymeric coating layer reduces or inhibits direct contact between ions in a liquid solution and the surface of the conductive substrate when the electrode is disposed in said liquid solution.

11. The electrode of any one of claims 1-10, wherein the polymeric coating layer has a porosity of about 10% (e.g., about 9%, about 8%, about 7%, about 6%, about 5%, about 4%, about 3% about 2.5%, about 2%, about 1.5%, about 1%, about 0.75%, about 0.5%) or lower.

12. The electrode of any one of claims 1-11, wherein the polymeric coating layer is substantially non-porous.

13. The electrode of any one of claims 1-12, wherein the polymeric coating layer completely covers the surface of the conductive substrate.

14. The electrode of any one of claims 1-13, wherein a thickness of the polymeric coating layer is about 95% or less of a distance between a target-binding portion or site of the target-binding molecule and the surface of the conductive substrate.

15. The electrode of any one of claims 1-14, wherein a thickness of the polymeric coating layer is at least about 1 nm.

16. The electrode of any one of claims 1-15, wherein a thickness of the polymeric coating layer is about 20 nm or lower.

17. The electrode of any one of claims 1-16, wherein the polymeric coating layer comprises an electropolymerized polymer, photopolymerized polymer, or auto-polymerized polymer.

18. The electrode of any one of claim 1-17, wherein the polymer coating layer comprises a electropolymerized polymer.

19. The electrode of any one of claim 1-18, wherein the polymeric coating layer comprises a polymer selected from the group consisting of polysaccharides, polydopamines, poly-p-phenylenes, polypyrroles, coumarin based polymers, polyhydroxyethylmethacrylates, polyindolyenes, polyanilines, polythiophenes, poly(ethylenedioxy) thiophene, poly(heteroaromatic vinylenes), polyvinylphosphates, poly-p-phenylene sulfides, poly(2,5-thienylenes), poly-o-phenylenes, poly(o-phenylene diamines), polysaccharides, polyacetylenes, porphyrins, phthalocyanins, fullerenes, polyparaphenylenes, polyphenylenevinylenes, polyfluorenes, polypyridines, polycarbazoles, polypyridinevinylenes, polyarylvinylenes, poly (p-phenylmethylvinylenes), derivatives and co-polymers thereof, and any combination thereof.

20. The electrode of any one of claims 1-19, wherein the polymeric coating layer comprises polydopamine, chitosan, or scopoletin (6-methoxy-7-hydroxycoumarin).

21. The electrode of any one of claim 1-20, wherein the polymeric coating layer comprises electropolymerized polydopamine, electropolymerized chitosan, electropolymerized scopoletin, or autopolymerized chitosan.

22. The electrode of any one of claim 1-21, wherein the polymeric coating layer further comprises a dissolvable or degradable material.

23. The electrode of claim 22, wherein the dissolvable or degradable material is selected from the group consisting of water dissolvable polymers (salt crystals), materials capable of being removed by degradation (e.g., proteins or protein aggregates), temperature dependent polymers (e.g., poly(N-isopropyl acrylamide) (PNIPAAm)); and any combination thereof.

24. The electrode of any one of claims 1-23, wherein the polymeric coating layer further comprises an antifouling material.

25. The method of claim 24, wherein the antifouling material is ethanolamine (ETA), hyaluronic acid (HA), or poly vinyl alcohol (PVA).

26. The electrode of any one of claim 1-25, wherein the target-binding molecule is a peptide, polypeptide, a nucleic acid (e.g., oligonucleotide or polynucleotide) or a small molecule.

27. The electrode of any one of claim 1-26, wherein the target binding molecule is a receptor, a ligand for a receptor, an antibody, antigen binding fragment of an antibody, an antigen, an enzyme, an affimer or an aptamer.

28. The electrode of one of claims 1-27, wherein the target binding molecule is an antibody, an antigen binding fragment of an antibody, or an antigen, optionally, the target binding molecule is an antibody or an antigen binding fragment of an antibody.

29. The electrode of any one of claims 1-28, wherein the electrode is a planar or 3-dimensional electrode.

30. The electrode of any one of claims 1-29, wherein the conductive substrate comprises gold, silver, copper, platinum, aluminum, stainless steel, tungsten, indium tin oxide, titanium, lead, nickel, palladium, zirconium, niobium, tantalum, chromium, molybdenum, manganese, rhenium, ruthenium, rhodium, iridium, silicon, polyimide, parylene, benzocyclobutene, carbon, graphite, or any combination thereof.

31. The electrode of any one of claims 1-30, wherein the conductive substrate comprises a flexible substrate.

32. The electrode of any one of claims 1-31, wherein the conductive substrate comprises a flexible substrate selected from the group consisting of polyethylene terephthalate, polyethylene naphathalate, polyimides, polymeric hydrocarbons, celluloses, plastics, polycarbonates, polystyrenes, silicon and any combination thereof.

33. The electrode of any one of claims 1-32, wherein the polymeric coating layer is adapted for contact with an analyte or a sample comprising an analyte.

34. The electrode of any one of claims 1-33, wherein the polymeric coating layer further comprises a conducting material.

35. The electrode of claim 34, wherein the conducting material comprises conductive particles, conductive rods, conductive fibers, conductive nano-particles, conductive polymers, conductive nano-flakes, conductive nanotubes, semi-conductive particles, semi-conductive rods, semi-conductive fibers, semi-conductive nano-particles, semi-conductive nanoflakes, semi-conductive nanotubes, or semi-conductive polymers.

36. The electrode of claim 34 or 35, wherein the conducting material is a metal, a metalloid, conducting polymer, a conducting carbon-based material, organic compounds having conducting and/or semiconducting properties, or any combination thereof.

37. The electrode of any one of claims 34-36, wherein the conducting material comprises an allotrope of carbon atoms arranged in a hexagonal lattice.

38. The electrode of any one of claims 34-37, wherein the conducting material is graphite, functionalised graphene oxide, reduced graphene oxide, or carbon nano-tubes (CNTs).

39. The electrode of claim 38, wherein the carbon nanotubes are carboxylated carbon nanotubes or aminated carbon nanotubes.

40. The electrode of claim 38, wherein the reduced graphene oxide is a carboxylated reduced graphene oxide or an aminated reduced graphene oxide.

41. The electrode of any one of claims 34-36, wherein the conductive material comprises gold.

42. The electrode of any one of claims 1-41, wherein the polymeric coating layer further comprises a redox active material.

43. The electrode of claim 42, wherein the redox active material is selected from the group consisting of metallocenes metallocence derivatives, quinones, polycyclic aromatic hydrocarbons, redox-active organic molecules, phenazines, organosulfur compounds, and radical acceptors.

44. The electrode of any one of claims 42 or 43, wherein the redox active material is selected from the group consisting of poly luminol, methylene blue, ferrocene, ferrocene derivatives, polyluminol, 3,3′,5,5′-tetramethylbenzidine (TMB), o-phenylenediamine dihydrochloride (OPD), N,N,N′,N′-tetramethyl-p-phenylenediamine, viologens 2,2′-azino-bis(3-ethylbenzothiazoline-6-sulfonic acid) (ABTS), p-nitrophenyl Phosphate (PNPP), 3,3′-diaminobenzidine (DAB), 4-chloro-1-naphthol (4-CN), 5-bromo-4-chloro-3-indolyl-phosphate (BCIP), nitro blue tetrazolium (NBT), tetrathialfulvaene, 2,6-dichloroindophenol (DCIP), 2,6-dichloroindophenyl phosphate, riboflavin 5′-monophosphate (RMP), ethyl viologen (1,1′-bis(ethyl)-4,4′-bipyridinium or BEBP), Paraquat, hexaamine ruthenium, 1,4-naphthoquione, resorufine, cyanomethylviologen, diquat, triquat, 1,1′-bis(2-sulfoethyl)-4,4′-bipyridinium, 1,1′-dibenzyl-4,4′-bipyridinium, 4,4′-dicarboxy-2,2′-bipyridyl, 1-hydroxybenzotriazole, veratryl alcohol, violuric acid, 2-methoxy-phenothiazone, 3-hydroxyanthranilic acid, anthraquinone 2,6-disulfonic acid, N-hydroxyacetanilide, phenol red, 3,3′,5,5′-tetramethyl benzidine, dichlorophenol red, 2,2′,6,6′-tetramethylpiperidine-N-oxyl radical, syringaldehyde and acetosyringone, 2,2,6,6-tetramethylpiperidin-1-yl)oxyl (TEMPO), 2,2′7,7′-tetrakis-(N,N-di-p-methoxyphenyl-amine)-9,9′-spirobifluorene (spiro-MeOTAD), sodium anthraquinone-2,6-di sulphonate (AQDS), benzoquinones, 2,2′-biimidazole, 2-(2-pyridyl)imidazole, 2,2′-bipyridine, tetracyanoquinodimethane (TCNQ), and combinations thereof.

45. The electrode of any one of claims 42-44, wherein the redox active material is poly luminol, ferrocene, or methylene blue.

46. The electrode of any one of claims 42-45, wherein the redox active material is embedded within the polymeric coating layer.

47. The electrode of any one of claims 1-46, wherein the target-binding molecule is covalently linked to the surface of the conductive substrate.

48. The electrode of any one of claims 1-47, wherein the target-binding molecule is linked to the surface of the conductive substrate via a linker.

49. A field-effect transistor (FET) comprising an electrode of any one of claims 1-48.

50. A sensor comprising an electrode of any one of claims 1-48 or a field-effect transistor of claim 49.

51. The sensor of claim 50, wherein the sensor comprises a fluid-contact surface and the electrode is immobilized on at least a portion of the fluid-contact surface.

52. The sensor of claim 50 or 51, wherein the sensor comprises one or more microfluidic flow cells.

53. The sensor of any one of claims 50-52, wherein the sensor comprises one or more open well-cells.

54. The sensor of any one of claims 50-53, wherein the fluid-contact surface further comprises a positive control electrode and/or a negative control electrode immobilized thereon.

55. Use of an electrode of any one of claims 1-48, or a field-effect transistor of claim 49, or a sensor of any one of claims 50-54 for detecting a target analyte in a sample.

56. A method for detecting a target analyte in a sample, the method comprising: contacting a sample suspected of comprising a target analyte with an electrode of any one of claims 1-48 and detecting binding of the target analyte with the target binding ligand.

57. The method of claim 56, wherein said detecting the binding of the target molecule with the target binding ligand comprises applying a voltage to the electrode.

58. The method of claim 56 or 57, wherein said detecting the binding of the target molecule with the target binding ligand comprises measuring a current generated from electrode.

59. The method of any one of claims 57 or 58, wherein said detecting the binding of the target molecule with the target binding molecule comprises contacting a second target binding molecule to the target molecule, wherein the second target binding molecule comprises a detectable label.

60. The method of claim 59, wherein said contacting with the second target binding molecule is prior to contacting the sample with the electrode.

61. The method of claim 59, wherein said contacting with the second target binding molecule is after contacting the sample with the electrode.

62. The method of any one of claims 59-61, wherein the detectable label comprises an enzyme, a fluorophore, a chemiluminescent label, colloidal gold, colored glass or plastic (e.g., polystyrene, polypropylene, and latex) beads, a radiolabel, a quantum dot, or any combination thereof.

63. The method of any one of claims 59-62, wherein the detectable label comprises an enzyme.

64. The method of claim 63, wherein the enzyme is a peroxidase, alkaline phosphatase, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase or acetylcholinesterase.

65. The method of any one of claims 63-64, wherein the method further comprises contacting the enzyme with a substrate of the enzyme.

66. The method of any one of claims 59-65, wherein the detectable label facilitates generation of a charge carrier.

67. The method of claim 66, wherein said detecting the binding of the target molecule with the target binding molecule comprises detecting the charge carrier.

68. The method of any one of claims 56-67, wherein the target analyte is a peptide, a polypeptide, a peptidomimetic, a nucleic acid, an oligosaccharide, a polysaccharide, an amino acid, nucleoside, a nucleotide, a carbohydrate, a lipid, a peptidoglycan, a cell, microbial matter, an antigen, a lipid, a steroid, a hormone, a lipopolysaccharide, an endotoxin, a therapeutic agent, a lipid-binding molecule, a cofactor, a small molecule, a toxin, a biological threat agent (e.g., spore, viral, cellular and protein toxin), or any combination thereof.

69. The method of any one of claims 56-68, wherein the target analyte is a protein, an antibody, an antigen binding fragment of an antibody, an antigen, a hormone, a nucleic acid, or a metabolite.

70. The method of any one of claims 56-69, wherein the target analyte is a tumor marker or a clinical chemistry target.

71. The method of any one of claims 56-70, wherein the sample is a biological sample (e.g., blood, saliva, amniotic fluid, sputum, urine, semen, cerebrospinal fluid, bronchial aspirate, perspiration, mucus, liquefied stool sample, synovial fluid, lymphatic fluid, tears, tracheal aspirate, lactation product, breadth condensate any combination thereof).

72. The method of any one of claims 56-71, wherein the sample is a food, an ingredient for preparing a food, poultry, meat, fish, beverage, food grain, a crop, or dairy product.

73. The method of any one of claims 65-71, wherein the sample is a non-biological sample.

74. The method of any one of claims 56-73, wherein the sample is pre-processed prior to contacting with the electrode or the sensor.

75. A kit comprising an electrode of any one of claims 1-48, or a field-effect transistor of claim 49, or a sensor of any one of claims 50-54.

76. A method for preparing an electrode comprising:
a. immobilizing a target binding molecule on a surface of a conductive substrate; and
b. forming or depositing a polymeric coating layer on said surface of the conductive substrate, and
wherein the polymeric coating layer covers at least a non-target binding portion of the target-binding molecule.

77. The method of claim 76, wherein a target-binding portion or site of the target-binding molecule is not covered by or embedded within the polymeric coating layer.

78. The method of claim 76 or 77, wherein a target-binding portion or site of the target-binding molecule is exposed for contact with an analyte, and wherein said contact with the analyte is not within the polymeric coating layer.

79. The method of any one of claims 76-78, wherein a target-binding portion or site of the target-binding molecule is at a surface of the polymeric coating layer.

80. The method of any one of claims 76-79, wherein a target-binding portion or site of the target-binding molecule is at a surface of the polymeric coating layer, and wherein said surface of the polymeric coating layer is not a surface of a pore in the polymeric coating layer.

81. The method of any one of claims 76-80, wherein the polymeric coating layer reduces or inhibits mobility ions in a liquid solution to the surface of the conductive substrate when the electrode is disposed in said liquid solution.

82. The method of any one of claims 76-81, wherein the polymeric coating layer reduces or inhibits direct contact between ions in a liquid solution and the surface of the conductive substrate when the electrode is disposed in said liquid solution.

83. The method of any one of claims 76-82, wherein the polymeric coating layer has a porosity of about 10% (e.g., about 9%, about 8%, about 7%, about 6%, about 5%, about 4%, about 3% about 2.5%, about 2%, about 1.5%, about 1%, about 0.75%, about 0.5%) or lower.

84. The method of any one of claims 76-83, wherein the polymeric coating layer is substantially non-porous.

85. The method of any one of claims 76-84, wherein the polymeric coating layer completely covers the surface of the conductive substrate.

86. The method of any one of claims 76-85, wherein a thickness of the polymeric coating layer is about 95% or less of a distance between a target-binding portion or site of the target-binding molecule and the surface of the conductive substrate.

87. The method of any one of claims 76-86, wherein a thickness of the polymeric coating layer is at least about 1 nm.

88. The method of any one of claims 76-87, wherein a thickness of the polymeric coating layer is about 20 nm or lower.

89. The method of any one of claims 76-88, wherein said forming or depositing the polymeric coating layer on the surface of the conductive substrate comprises electro-polymerization, photo-polymerization, or auto-polymerization, optionally, said forming or depositing the polymeric coating layer on the surface of the conductive substrate comprises electro-polymerization.

90. The method of any one of claims 76-89, wherein the polymeric coating layer comprises a polymer selected from the group consisting of polysaccharides, polydopamines, poly-p-phenylenes, polypyrroles, coumarin based polymers, polyhydroxyethylmethacrylates, polyindolyenes, polyanilines, polythiophenes, poly(ethylenedioxy) thiophene, poly(heteroaromatic vinylenes), polyvinylphosphates, poly-p-phenylene sulfides, poly(2,5-thienylenes), poly-o-phenylenes, poly(o-phenylene diamines), polysaccharides, polyacetylenes, porphyrins, phthalocyanins, fullerenes, polyparaphenylenes, polyphenylenevinylenes, polyfluorenes, polypyridines, polycarbazoles, polypyridinevinylenes, polyarylvinylenes, poly (p-phenylmethylvinylenes), derivatives and co-polymers thereof, and any combination thereof.

91. The method of any one of claims 76-90, wherein the polymeric coating layer comprises polydopamine, chitosan, or scopoletin (6-methoxy-7-hydroxycoumarin).

92. The method of any one of claims 76-91, wherein said immobilizing the target binding molecule on the surface of the conductive substrate comprises covalently linking the target binding molecule on the surface of the conductive substrate.

93. The method of any one of claims 76-92, wherein said immobilizing the target binding molecule on the surface of the conductive substrate comprises linking the target binding molecule on the surface of the conductive substrate via a linker.

94. The method of any one of claims 76-93, wherein the target-binding molecule is a peptide, polypeptide, a nucleic acid (e.g., oligonucleotide or polynucleotide) or a small molecule.

95. The method of any one of claims 76-94, wherein the target binding molecule is a receptor, a ligand for a receptor, an antibody, antigen binding fragment of an antibody, an antigen, an enzyme, an affimer or an aptamer.

96. The method of any one of claims 76-95, wherein the target binding molecule is an antibody, an antigen binding fragment of an antibody, or an antigen, optionally, the target binding molecule is an antibody or an antigen binding fragment of an antibody.

97. The method of any one of claims 76-96, further comprising adding an antifouling material to the polymeric coating layer.

98. The method of claim 97, wherein said adding the antifouling material to the polymeric coating layer is prior to forming or depositing the polymeric layer on the surface of the conductive substrate.

99. The method of claim 97 or 98, wherein the antifouling material is ethanolamine, hyaluronic acid, or poly vinyl alcohol.

100. The method of any one of claims 76-99, wherein the electrode is a planar or 3-dimensional electrode.

101. The method of any one of claims 76-100, wherein the conductive substrate comprises gold, silver, copper, platinum, aluminum, stainless steel, tungsten, indium tin oxide, titanium, lead, nickel, palladium, zirconium, niobium, tantalum, chromium, molybdenum, manganese, rhenium, ruthenium, rhodium, iridium, silicon, polyimide, parylene, benzocyclobutene, carbon, graphite, or any combination thereof.

102. The method of any one of claims 76-101, wherein the conductive substrate comprises a flexible substrate.

103. The method of any one of claims 76-102, wherein the conductive substrate comprises a flexible substrate selected from the group consisting of polyethylene terephthalate, polyethylene naphathalate, polyimides, polymeric hydrocarbons, celluloses, plastics, polycarbonates, polystyrenes, silicon and any combination thereof.

104. The method of any one of claims 76-103, wherein the polymeric coating layer is adapted for contact with an analyte or a sample comprising an analyte.

105. The method of any one of claims 76-104, wherein the electrode is an electrode of any one of claims 1-48.
